(19) United States
(12) Patent Application Publication
(10) Pub. No.: US 2016/0131634 A1
(43) Pub. Date: May 12, 2016

(54) METHODS OF EVALUATING CELL CULTURE ADDITIVES

(71) Applicant: BIOGEN MA INC., Cambridge, MA (US)

(72) Inventors: Weiwei Hu, Cary, NC (US); Haofan Peng, Cary, NC (US); Erik Hughes, Raleigh, NC (US); Kelly Wiltberger, Durham, NC (US); Maureen Lanan, Newton, MA (US); Amr Ali, Medford, MA (US)

(73) Assignee: Biogen MA Inc., Cambridge, MA (US)

(21) Appl. No.: 14/892,723

(22) PCT Filed: May 29, 2014
(86) PCT No.: PCT/US14/40088

§ 371 Date: Nov. 20, 2015

Related U.S. Application Data
(60) Provisional application No. 61/828,603, filed on May 29, 2013.
(60) Provisional application No. 61/897,864, filed on Oct. 31, 2013.

(51) Int. Cl. G01N 33/483

(52) U.S. Cl. CPC G01N 33/4833

(57) ABSTRACT

The present disclosure shows, unexpectedly, that variations in cell culture performance in large-scale cell culture systems such as, for example, those used in commercial manufacturing processes, in some instances, can be attributed to often subtle variations among shear-protectant additives used during cell culture. Assessing the quality of shear-protective additives using such large-scale systems, however, is inaccurate, time-consuming and costly. To solve the problem identified, the present disclosure provides methods and compositions for evaluating the suitability of shear-protectant additives without resorting to large scale cell growth and/or protein production tests.

### RELATED APPLICATIONS

This application claims the benefit under 35 U.S.C. §119(e) of U.S. provisional application No. 61/828,603, filed May 29, 2013, and of U.S. provisional application No. 61/897,864, filed Oct. 31, 2013, each of which is incorporated by reference herein in its entirety.

### FIELD OF THE INVENTION

The present disclosure, in some embodiments, relates to the evaluation of variations in cell culture additives.

### BACKGROUND OF THE INVENTION

Stirred tank bioreactors with gas sparging are typically used for large-scale mammalian cell culture in commercial manufacturing processes. To prevent cell shear damage associated with the gas bubbles that are introduced to the cell culture system by sparging, additives such as, for example, nonionic surfactants (e.g., poloxamers) are often used. Nonionic surfactants prevent cell damage by associated air bubbles and this, in turn, increases cell growth and viability. Nonetheless, even with the use of nonionic surfactants and other shear-protectant additives, cell viability and viable cell density vary among cell culture batches, even in the same facility using the same manufacturing equipment.

### SUMMARY OF THE INVENTION

The present disclosure shows, unexpectedly, that variations in cell culture performance in large-scale cell culture systems such as, for example, those used in commercial manufacturing processes, in some instances, can be attributed to often subtle variations among shear-protectant additives used during cell culture. Assessing the quality of shear-protective additives using such large-scale systems, however, is inaccurate, time-consuming and costly. To solve the problem identified, the present disclosure provides methods and compositions for evaluating the suitability of shear-protectant additives without resorting to large scale cell growth and/or protein production tests. In some embodiments, shear-protectant compositions can be evaluated by analyzing their molecular weight and/or hydrophobicity properties. In some embodiments, suspicious lots of shear-protectant can be identified if they have high molecular weight components and/or highly hydrophobic components that are not present in shear-protectant lots that are effective for cell growth and/or protein productions. In some embodiments, simple and efficient small-scale systems such as, for example, shake flask (e.g., baffled shake flask) systems, can be used to assess variations in the quality among shear-protectant additives (e.g., among different batches of additives). These and other methods are described in more detail herein.

Surprisingly, the present disclosure shows that, in some embodiments, the presence or absence of highly hydrophobic components and/or high molecular weight components in samples of shear-protectant additives is indicative of efficacy of the additive for preventing shear damage. Shear-protectant additives (e.g., particular lots, or batches, of shear-protectant additives) that are effective for preventing cellular shear damage, for example, in large-scale cell culture systems are referred to herein as “suitable” additives (also referred to herein as “good” additives). Shear-protectant additives that are ineffective for preventing cellular shear damage, or that are not as effective as a suitable shear-protective additive, are referred to herein as “unsuitable” additives (also referred to herein as “bad” or “suspicious” additives). In some embodiments, use of an unsuitable shear protectant additive results in reduced cell viability, reduced cell density and/or reduced protein titer (e.g., when used in cell culture systems for protein manufacturing processes). Shear-protectant additives that are less effective than a suitable additive and more effective than an unsuitable additive are referred to herein as “intermediate” additives. It should be appreciated that in some embodiments, a shear-protectant composition may be identified as suspicious if it has one or more properties (e.g., hydrophobicity and/or molecular weight profiles) that are characteristic of an unsuitable shear-protectant even if the suspicious shear-protectant has not been evaluated in a cell growth assay.

Accordingly, methods and compositions described herein can be used to evaluate a shear-protectant composition to determine whether it is suitable for use in a cell growth and/or protein production procedure. In some embodiments, a lot or batch of a shear-protectant that has at least one property that is characteristic of an unsuitable shear-protectant is not used, for example, is excluded from a commercial cell growth and/or protein production procedure. A shear-protectant can be evaluated in any form that can be analyzed, for example, in the form of a powder, a solution, or any other form that can be analyzed to determine the presence of one or more properties that are characteristic of an unsuitable shear-protectant.

It should be appreciated that polymeric shear-protectant compositions can comprise a distribution of different polymers (e.g., having different sizes and/or relative content of the polymer components). In some embodiments, a polymeric shear-protectant composition is evaluated to determine whether it contains a distribution of polymers that is similar to (a) a composition that is known to be suitable for cell growth and/or protein production (e.g., on a large scale, for example in a manufacturing scale fermenter), and/or (b) a composition that is known to be unsuitable for cell growth and/or protein production. In some embodiments, a shear-protectant composition is evaluated to determine whether it contains highly hydrophobic components and/or high molecular weight components in an amount that is (a) different from (e.g., statistically higher than) an amount characteristic of a known suitable shear-protectant, and/or (b) similar (e.g., statistically significantly similar) to an amount characteristic of a known unsuitable shear-protectant.

In some embodiments, the hydrophobicity of a shear-protectant composition is evaluated (e.g., measured or determined) without fractionating the composition and/or without isolating certain components from the composition. However, in some embodiments, the hydrophobicity of one or more fractions of the shear-protectant composition is evaluated. For example, in some embodiments one or more fractions having different molecular weight ranges are evaluated.

In some embodiments, the molecular weight profile of a shear-protectant composition is evaluated (e.g., measure or determined). In some embodiments, the relative amount of one or more high molecular weight components present in a shear-protectant composition can be evaluated by determining the relative amount of one or more high molecular weight fractions in the composition. In some embodiments, the relative amount of high molecular weight components in a shear-protectant composition being evaluated is determined relative to a suitable reference (e.g., the total amount of material in the composition, the amount of material having an average molecular weight of the composition, the amount of one or more lower molecular weight fractions of the composition, or other suitable reference). In some embodiments, the amount of shear-protectant material in one or more high molecular weight fractions (e.g., the highest 5%, 10%, 15%, 20%, 25%, 30% or 35% of the molecular weight range of the shear-protectant composition being evaluated) is determined and compared to (e.g., divided by) a suitable reference amount of material for the composition being evaluated. In some embodiments, a shear-protectant composition is identified as suspicious if it contains an amount of high molecular weight material that is higher (e.g., statistically higher) than a suitable composition. In some embodiments, the high molecular weight material is identified as a particular peak in a molecular weight profile. In some embodiments, the high molecular weight material is identified as one or more peaks above a particular reference molecular weight. However, in some embodiments, the presence of a suspicious amount of a high molecular weight material can result in a change in the overall distribution (e.g., the presence of a shoulder or bump in the higher molecular weight fractions of the molecular weight distribution of a composition being evaluated indicating the presence of a higher than expected amount of high molecular weight material even if one or more discrete peaks are not identified).

In some embodiments, the shear-protective additive is poloxamer 188 (e.g., PLURONIC®, KOLLIPHOR® or LUTROL®). A high molecular weight (HMW) component detected in a sample of an unsuitable lot of poloxamer 188, for example, may have a molecular weight of at least 12,000 Daltons. For example, a HMW component detected in a sample of an unsuitable lot of poloxamer 188 may have a molecular weight of at least 12.5 kilodaltons (kDA), at least 13 kDa, at least 13.5 kDa, or at least 14 kDa.

In some embodiments, an unsuitable sample of poloxamer 188 contains high molecular weight components that, when assessed by size exclusion chromatography (SEC), elute at 12 to 13.5 minutes into an SEC run, represented by a HMW peak in a chromatogram that has an area of greater than 0.03%, greater than 0.04% or greater than 0.05% of the total area of the chromatogram. This HMW peak percentage is based, in some embodiments, on the integration of that peak with the respect to the integral of the entire peak (e.g., main peak) of the shear-protectant additive (e.g., the area of the peaks can be calculated using Waters Empower 2.0 Chromatography Data Software). Conversely, in some embodiments, a suitable sample of poloxamer 188 does not contain high molecular weight components that, when assessed by SEC, elute at 12 to 13.5 minutes into an SEC run, represented by a HMW peak in a chromatogram that has an area of greater than 0.05% of the total area of the chromatogram. In some embodiments, if a HMW peak is produced in a chromatogram of a suitable sample of poloxamer 188 at a time between 12 to 13.5 minutes, the HMW peak has an area of less than 0.05% of the total area of the chromatogram. In some embodiments, the HMW peak of a suitable shear-protective additive is less than 0.04% or less than 0.03% of the entire chromatogram. In some embodiments, the amount of a HMW peak is compared to the amount of that peak in a known suitable or unsuitable shear-protectant composition (e.g., to determine whether it is statistically higher or similar, respectively, relative to the amount in the known suitable or unsuitable composition).

For example, FIG. 16, top panel, shows a chromatogram of a suitable sample of poloxomer 188 (“high performance lot”). The area of Peak 1, representative of HMW components eluting between 12 and 13.5 minutes into the SEC run, is less than 0.05% of the area of the Main Peak, representative of components eluting between 14.5 minutes and 17.5 minutes into the SEC run. FIG. 16, middle and bottom panels, shows chromatograms of an unsuitable sample of poloxomer (“medium performance lot” and “low performance lot”). The area of Peak 1 in each chromatogram, representative of HMW components eluting between 12 and 13.5 minutes into the SEC run, is greater than 0.05% of the area of the Main Peak, representative of components eluting between 14.5 minutes and 17.5 minutes into the SEC run.

In some embodiments, an unsuitable shear-protective additive contains highly hydrophobic components. For example, when assessing efficacy of various samples of poloxamer 188, an unsuitable sample (e.g., a batch or preparation, for example a liquid batch or preparation of the poloxamer) may have a hydrophilic-lipophilic balance (HLB) value of less than 29. In some embodiments, an unsuitable sample may have a HLB value of less than 28, less than 27, less than 26, less than 25, less than 24, less than 23, less than 22 less than 21 or less than 20. In some embodiments, an unsuitable sample may have a HLB value of 10 to 28. Conversely, in some embodiments, a suitable sample of poloxamer 188 does not contain highly hydrophobic components.

In some embodiments, an unsuitable shear-protectant additive contains highly hydrophobic components that have a high molecular weight. For example, an unsuitable sample of poloxamer 188 may contain components having a molecular weight of at least 12 kDA (e.g., at least 12.5 kDA, at least 13 kDA, at least 13.5 kDA, at least 14 kDA, or at least 14.5 kDA) and have a HLB value of less than 29.

The effects of a shear-protectant additive on various cell performance parameters (e.g., cell viability, viable cell density), which, in some embodiments, are indicators of suitable and unsuitable shear-protectant additives, can be assessed directly or indirectly. A method is considered to “directly” assess efficacy of a shear-protectant additive if the method includes the use of viable cells, for example, to assess one or more of various cell performance parameters. Thus, in some embodiments, small-scale methods provided herein are useful for comparing cell performance values associated with different lots of the same type of shear-protectant additive (e.g., different lots of the same poloxamer) in order to select a lot that is suitable for large-scale cell culture manufacturing processes (e.g., manufacturing therapeutic proteins such as antibodies). A method is considered to “indirectly” assess efficacy of a shear-protectant additive if the method does not include the use of viable cells. For example, presence of high molecular weight components and/or highly hydrophobic components in a sample of a shear-protectant additive may be indicative that the additive is an unsuitable shear-protectant additive.

The present disclosure also provides, inter alia, various small-scale methods for assessing efficacy of shear-protectant additives for large-scale cell culture systems. For example, the effects of bioreactor sparging on cells during culture can be replicated by carefully generating in solution (e.g., cell culture media) a sufficient amount of bubbles of adequate size, which form a “foam layer” of the solution. Surprisingly, the stability of a foam layer produced by agitation of a solution containing a sample of a shear-protectant additive in a shake flask (e.g., baffled shake flask) correlates with efficacy of the additive. Also surprising is the presence of a high molecular weight components present in the foam layer, which is indicative that the additive is unsuitable

Aspects of the present disclosure provide methods for evaluating efficacy of a shear-protectant additive for preventing shear damage to cells. In some embodiments, methods comprise detecting in a sample of a shear-protectant additive a high molecular weight component and/or a highly hydrophobic components, and identifying the sample as an unsuitable sample. In some embodiments, the shear-protectant additive is poloxamer 188 and the high molecular weight component has a molecular weight of greater than 12,000 Daltons. In some embodiments, the shear-protectant additive is poloxamer 188 that has a hydrophilic-lipophilic balance (HLB) value of less than 29. In some embodiments, methods comprise assaying a sample of a shear-protectant additive for a high molecular weight component and/or a highly hydrophobic components, and identifying the sample as a suitable sample if a high molecular weight components and/or a highly hydrophobic components is not detected.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Generally, the two hydrophilic chains of polyoxyethylene constitute 80% of the copolymer. In some instances, however, the proportion of hydrophilic chains constitutes less than 80% of the copolymer. The present disclosure shows that the proportion of hydrophilic chains and hydrophobic chains can be indicative of efficacy of a poloxamer (e.g., solution of poloxamer) for protecting against cell shear damage. Thus, in some embodiments, methods of the present disclosure comprise determining the proportion of hydrophilic chains and hydrophobic chains in poloxamer copolymers obtained from a sample of a poloxamer (e.g., a solution containing poloxamer 188), and then identifying the sample as unsuitable if the hydrophilic chains constitutes less than 80% of the copolymers. In some embodiments, the methods comprise identifying the sample as unsuitable if the hydrophilic chains constitute less than 78%, less than 75% or less than 70% of the copolymers.

Generally, shear-protectant additives (also referred to herein as shear-protectant compositions) of the present disclosure are surfactants, which contain a distribution of different surface active components, including a mixture of polymers having different molecular weights. “Components” or “species” (used interchangeably) of the additives (or compositions) of the present disclosure refers to polymers in the additives. Thus, a “poloxamer component” refers to a polymer among a mixture of polymers having different molecular weights.

In some embodiments, a sample of a shear-protectant additive is assayed for high molecular weight components using size exclusion chromatography (SEC). In some embodiments, a sample of a shear-protectant additive is assayed for hydrophobic and/or hydrophilic components using reverse-phase high performance liquid chromatography (RP-HPLC).

Aspects of the present disclosure provide methods for evaluating sample variations (e.g., lot-to-lot variations) of a shear-protectant additive (e.g., poloxamer 188). In some embodiments, methods may comprise the steps of (a) producing, in a solution that comprises viable cells and a shear-protectant additive at a concentration of 0.01 g/L to 10 g/L solution, bubbles in an amount sufficient to cause a greater than 5% drop in cell viability relative to initial cell viability, (b) measuring one or more cell performance parameters of the cells to obtain one or more cell performance values, and (c) selecting the shear-protectant additive if the one or more cell performance values is comparable to one or more reference values. It should be understand that “an amount sufficient to cause a greater than 5% drop in cell viability relative to initial cell viability” in a solution refers to an amount of bubbles that would cause a greater than 5% drop in cell viability relative to initial cell viability if a shear protectant additive was otherwise excluded from the solution. Generally, the presence of a suitable shear-protectant additive in a solution of viable cells reduces the drop in cell viability (e.g., by a percentage as specified herein) relative to a solution without the suitable shear protectant additive and/or relative to a solution with an unsuitable shear-protectant additive. The presence of an unsuitable shear-protectant additive in a solution of viable cells (1) does not reduce the drop in cell viability (e.g., by a percentage as specified herein) relative to a solution without the unsuitable shear-protectant additive, or (2) reduces the drop in cell viability to a lesser extent relative to a solution with a suitable shear protectant additive.

In some embodiments, methods comprise the steps of (a) producing, in a solution that comprises viable cells and a shear-protectant additive at a concentration of 0.01 g/L to 10 g/L solution, bubbles in an amount sufficient to cause a greater than 5% drop in cell viability relative to initial cell viability, (b) measuring the viability of the cells, and (c) selecting the shear-protectant additive if the viability of the cells drops by less than 10% as compared to the initial cell viability.

In other embodiments, methods comprise the steps of, for each of a plurality of shear-protectant additives, (a) producing, in a solution that comprises viable cells and a shear-protectant additive at a concentration of 0.01 g/L to 10 g/L solution, bubbles in an amount sufficient to cause a greater than 5% drop in cell viability relative to initial cell viability, (b) measuring the viability of the cells, and (c) selecting the shear-protectant additive if the viability of the cells is greater than 80%.

In still other embodiments, methods comprise the steps of (a) producing, in a first solution that comprises viable cells and a shear-protectant additive at a concentration of 0.01 g/L to 10 g/L solution, bubbles in an amount sufficient to cause a greater than 5% drop in cell viability relative to initial cell viability, (b) producing, in a second solution that comprises viable cells and a shear-protectant additive at a concentration of 0.01 g/L to 10 g/L solution, bubbles in an amount sufficient to cause a greater than 5% drop in cell viability relative to initial cell viability, (c) measuring one or more cell performance parameters of the cells in the first and second solution, and (d) selecting the shear-protectant additive that is most effective for protecting cells against shear damage. For example, a first shear-protectant additive is more effective than a second shear-protective additive if the first shear-protectant additive reduces the drop in cell viability to a greater extent relative to the second shear-protective additive. Likewise, a first shear-protectant additive is more effective than a second shear-protective additive if the first shear-protectant additive increases cell viability to a greater extent relative to the second shear-protective additive.

In some embodiments, methods further comprise shaking the solution in a shake flask. The shake flask may be a baffled shake flask. In some embodiments, the shake flask may have a volume of less than 10 L, 125 ml to 3 L, or 1 L.

In some embodiments, the working volume of the solution in the shake flask is 10% to 30% of the volume of the shake flask.

In some embodiments, the solution comprises water, buffer and/or cell culture media.

In some embodiments, the shear-protectant additive is a surfactant. For example, the shear-protectant additive may be a poloxamer, a polyvinyl alcohol or a polyethylene glycol. In some embodiments, the surfactant is a poloxamer. Non-limiting examples of poloxamers for use as provided herein include PLURONIC®, KOLLIPHOR® and LUTROL®.

In some embodiments, the concentration of the shear-protectant additive is 0.5 g/L to 2 g/L solution.

In some embodiments, cells are mammalian cells.

In some embodiments, methods further comprise culturing viable cells in the solution. For example, the cells may be cultured for 15 minutes to 1 week.

In some embodiments, cells are cultured at a temperature of 30° C. to 40° C.

In some embodiments, cells are cultured at a CO₂ concentration of 3% to 10%.

Other aspects of the present disclosure provide methods for evaluating sample variations of a shear-protectant additive by (a) producing a foam layer in a solution that comprises a shear-protectant additive at a concentration of 0.01 g/L to 10 g/L solution, (b) measuring a duration of time during which the foam layer dissipates, and (c) selecting the shear-protectant additive if the duration of time during which the foam layer dissipates is comparable to a reference value. In some embodiments, the reference value is a pre-determined value. In some embodiments, the reference value is based on a dissipation time from (e.g., obtained from) a control sample of a solution containing a sample of a shear-protectant additive known to be effective for protecting cells against shear damage, referred to herein as a suitable shear-protectant additive. In some embodiments, the solution is a cell-free solution. Also provided herein are methods that comprise the steps of (a) producing a foam layer in a first solution that comprises a first shear-protectant additive at a concentration of 0.01 g/L to 10 g/L solution, (b) producing a foam layer in a second solution that comprises a second shear-protectant additive at a concentration of 0.01 g/L to 10 g/L solution, (c) measuring a duration of time during which the foam dissipates in the first and second solutions to obtain a first and second dissipation time, respectively, and (d) selecting the shear-protectant additive with the shortest dissipation time. In some embodiments, the solution is a cell-free solution.

In some embodiments, the solution further comprises an antifoaming agent (also referred to as a defoaming agent).

In some embodiments, methods comprise the steps of (a) producing a foam layer in a test solution that comprises a sample of shear-protectant additive at a concentration of 0.01 g/L to 10 g/L test solution, (b) collecting a liquefied foam layer sample from the test solution, (c) producing a size exclusion chromatography (SEC) chromatogram of the liquefied foam layer sample, (d) comparing the high molecular weight peak of the SEC chromatogram to a reference value, and (e) selecting the shear-protectant additive if the high molecular weight peak of the SEC chromatogram is comparable to the reference value. In some embodiments, the reference value is a pre-determined value. In some embodiments, the reference value is based on a high molecular weight peak of a SEC chromatogram from (e.g., obtained from) a control sample of a solution containing a sample of a shear-protectant additive known to be effective for protecting cells against shear damage, referred to herein as a suitable shear-protectant additive. In some embodiments, the control sample is from the bulk layer (e.g., non-foam layer) of the test solution. The foam layer is highly enriched in hydrophobic components relative to the bulk layers. In some embodiments, the test solution is a cell-free solution.

In some embodiments, methods comprise the steps of (a) producing a foam layer in a first test solution that comprises a first sample of shear-protectant additive at a concentration of 0.01 g/L to 10 g/L test solution, (b) producing a foam layer in a second test solution that comprises a second sample of shear-protectant additive at a concentration of 0.01 g/L to 10 g/L test solution, (c) collecting first and second liquefied foam layer samples from the first and second test solutions, respectively, (d) producing a first and second size exclusion chromatography (SEC) chromatogram of the first and second liquefied foam layer samples, respectively, (e) comparing the high molecular weight peak of the first and second SEC chromatograms to each other, and (f) selecting the shear-protectant additive having the smallest high molecular weight peak (e.g., high molecular weight peak having the shortest height (and/or smallest area) along the y-axis of a standard chromatogram). In some embodiments, the second test solution comprises a control solution containing a sample of a shear-protectant additive known to be effective for protecting cells against shear damage, referred to herein as a suitable shear-protectant additive. In some embodiments, the test solution is a cell-free solution.

In some embodiments, methods comprise the steps of (a) producing a foam layer in a plurality of test solutions that each comprise a sample of respective shear-protectant additives at a concentration of 0.01 g/L to 10 g/L test solution, (b) collecting a liquefied foam layer sample from respective test solutions, (c) producing a size exclusion chromatography (SEC) chromatogram of respective liquefied foam layer samples, (d) comparing the high molecular weight peaks of respective SEC chromatograms, and (e) selecting the shear-protectant additive with the smallest high molecular weight peak. In some embodiments, the test solution is a cell-free solution.

In some embodiments, the volume of the foam layer is 20% to 200% of the total volume of the solution. For example, the volume of the foam layer may be 100% of the total volume of the solution.

In some embodiments, methods further comprise shaking the solution in a shake flask. The shake flask may be a baffled shake flask. In some embodiments, the shake flask may have a volume of less than 10 L, 125 ml to 3 L, or 1 L.

In some embodiments, the working volume of the solution in the shake flask is 10% to 30% of the volume of the shake flask.

In some embodiments, the solution comprises water, buffer and/or cell culture media.

In some embodiments, the shear-protectant additive is a surfactant. For example, the shear-protectant additive may be a poloxamer, a polyvinyl alcohol or a polyethylene glycol. In some embodiments, the surfactant is a poloxamer.

In some embodiments, the concentration of the shear-protectant additive is 0.5 g/L to 2 g/L solution.

In some embodiments, the cells are mammalian cells.

In some embodiments, methods further comprise culturing the viable cells in the solution. For example, the cells may be cultured for 15 minutes to 1 week.

In some embodiments, the cells are cultured at a temperature of 30° C. to 40° C. In some embodiments, the cells are cultured at a CO₂ concentration of 3% to 10%. However, in some embodiments, the cells are not cultured in the solution prior to performing the assay.

In some embodiments, the reference value is a dissipation time obtained from a control solution containing a shear-protectant additive effective for protecting cells against shear damage.

In some embodiments, the reference value is 40 minutes, and the shear-protectant additive is selected if the dissipation time is less than 40 minutes. In some embodiments, the reference value is 30 minutes, and the shear-protectant additive is selected if the dissipation time is less than 30 minutes. In some embodiments, the reference value is 20 minutes, and the shear-protectant additive is selected if the dissipation time is less than 20 minutes.

These and other aspects of the invention are described in more detail herein.

The invention is not limited in its application to the details of construction and the arrangement of components set forth in the following description or illustrated in the drawings. The invention is capable of other embodiments and of being practiced or of being carried out in various ways. Each of the above embodiments and aspects may be linked to any other embodiment or aspect. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. For purposes of clarity, not every component may be labeled in every drawing.

FIG. 1 shows a non-limiting example of a graph plotting viable cell density (VCD) as a function of time (top) and a graph plotting cell viability as a function of time (bottom). The data was collected from large-scale bioreactor cell cultures using cell culture media supplemented with shear-protectant additive, PLURONIC® F-68 (lot S1);

FIG. 2A shows a non-limiting example of a graph plotting normalized viable cell density (top) and a graph plotting cell viability drop (bottom) for small-scale baffled shake flask cell cultures using cell culture media supplemented with a sample from respective lots of PLURONIC® F-68. The cells were cultured for a period of 3 days. FIG. 2B shows that the difference in viability drop between suitable and unsuitable PLURONIC® F-68 lots can be observed as quickly as 15 minutes;

FIG. 3 shows a non-limiting example of a graph plotting normalized viable cell density (top) and a graph plotting cell viability drop (bottom) for small-scale baffled shake flask cell cultures using cell culture media supplemented with a sample from respective lots of PLURONIC® F-68. The cells were cultured for a period of 1 day;

FIG. 4 shows a non-limiting example of a graph plotting normalized viable cell density for small-scale baffled shake flask cell cultures using cell culture media supplemented with a sample from respective lots of PLURONIC® F-68 for each of three different cell lines;

FIG. 5 shows a non-limiting example of a graph plotting viable cell density as a function of time (top) and a graph plotting cell viability as a function of time (bottom). The data was collected from large-scale bioreactor cell cultures using cell culture media supplemented with a sample from a shear-protectant additive, PLURONIC® F-68 (lot N6, FIGS. 2 and 3);

FIG. 6 shows a non-limiting example of a graph plotting static surface tension data of samples from respective lots of PLURONIC® F-68 measured by a pendant drop method. 7, 18: suitable/good lots; 3, 15, 19: unsuitable/suspicious lots; 11: intermediate lot; 1, 2, 4-6, 8-10, 12-14, 16, 17, 21, 21: unknown lots;

FIG. 7 shows a non-limiting example of photographs of foam generated after shaking in a baffled shake flask containing PLURONIC® F-68 and an antifoaming agent (left) and a control (unbaffled) shake flask containing PLURONIC® F-68 and an antifoaming agent (right);

FIG. 8 shows a non-limiting example of graphs comparing foam dissipation times (also referred to as defoam times) among samples from respective lots of PLURONIC® F-68 (left) and viability drop in cell culture tests among the same lots (right);

FIG. 9 shows a non-limiting example of graphs comparing foam dissipation times among samples from respective lots of PLURONIC® F-68 (left) and viability drop in cell culture tests among the same lots (right);

FIG. 10 shows a non-limiting example of graphs comparing foam dissipation times among samples from respective lots of PLURONIC® F-68 (left) and viability drop in cell culture tests among the same lots (right);

FIG. 11A shows a non-limiting example of a composite graph of the data presented in the graphs of FIGS. 11B-11F. FIGS. 11B and 11C show size exclusion chromatography (SEC) chromatograms of bulk liquid samples and liquefied foam layer samples produced using samples from unsuitable/suspicious lots of PLURONIC® F-68. Peaks are located in high molecular weight regions. FIG. 11D shows an SEC chromatogram of a bulk liquid sample and liquefied foam layer sample produced using a sample from an unsuitable (“intermediate”) lot of PLURONIC® F-68. FIGS. 11E and 11F show SEC chromatograms of bulk liquid samples and liquefied foam layer samples produced using samples from suitable (“good”) lots (or control lots) of PLURONIC® F-68; and

FIG. 12A shows a non-limiting example of a composite graph of the data presented in the graphs of FIGS. 12B-12E. FIGS. 12B and 12C show size exclusion chromatography (SEC) chromatograms of bulk liquid samples and liquefied foam layer samples produced using unsuitable/suspicious lots of PLURONIC® F-68. Peaks are located in high molecular weight regions. FIGS. 12D and 12E show SEC chromatograms of bulk liquid samples and liquefied foam layer samples produced using suitable lots of PLURONIC® F-68.

FIG. 13 shows a graph illustrating the effect on cell growth of adding a small amount of a highly hydrophobic molecule to a suitable shear-protectant additive.

FIG. 14A shows a chromatogram obtained from a reverse phase-high performance liquid chromatography (RP-HPLC) analysis of SEC fractions obtained from an unsuitable lot of a shear-protectant additive. FIG. 14B shows a chromatogram obtained from a RP-HPLC analysis of SEC fractions obtained from a suitable lot of a shear-protectant additive.

FIG. 15 shows SEC chromatograms of samples of a suitable shear-protectant additive (top panel) and unsuitable shear protectant additives (middle and bottom panels). Peak 1, present between 12 and 13.5 minutes, is indicative of efficacy of the additive of preventing shear damage to cells.

### DETAILED DESCRIPTION OF THE INVENTION

Various aspects and embodiments of the present disclosure are directed to small-scale methods for evaluating sample (e.g., batch-to-batch) variations of a shear-protectant additive, for example, for use in large-scale manufacturing processes (e.g., a cell-culture based manufacturing process). Small-scale methods of the present disclosure provide cost-effective and efficient ways, without the use of costly and time-consuming large-scale sparged bioreactor cell culturing, to evaluate the effectiveness of shear-protectant additives. This can be achieved, in some embodiments, by evaluating the hydrophobicity and/or molecular weight profiles of shear-protectant compositions. In other embodiments, the effectiveness of a shear-protectant can be evaluated in small scale cell culture systems described herein. For example, this can be achieved, in some embodiments, in the absence of sparging by introducing air bubbles into a small-scale system, with or without viable cells, through agitation of solution in vessels with a volume of less than 10 L (e.g., less than 1 L, for example about 125 ml, 250 ml, or 500 ml). For example, in some embodiments, baffled shake flasks are used, which unexpectedly mimic a large-scale cell culture environment in which cell shear damage occurs.

Provided herein are assays that can be used to directly assess the effectiveness of a sample of shear-protectant additive on protecting cells from shearing, or shear damage. Such “direct” methods include viable cells in solution, whereby the viability of the cells is directly assessed in the presence of a sample of a shear-protective additive. Also provided herein are assays that can be used to indirectly assess the effectiveness of a sample of shear-protectant additive on protecting cells from shear damage. Such “indirect” methods are typically cell-free (i.e., do not include viable cells), and thus do not directly assess cell viability. Rather, such indirect methods, based on the results of the assay, permit a correlation to be made with respect to the effectiveness of the shear-protective additive. Thus, methods provided herein may be used to assess what may be referred to herein as a “test sample” of a shear-protectant additive. In some embodiments, a test sample of a shear-protectant additive is obtained from a new lot or batch of additive that has not yet been assessed for its effectiveness in preventing cell shear damage.

It should be appreciate that, in some embodiments, indirect methods, which are typically cell-free, may, in some instances, include cells. For example, it may be possible to combine direct and indirect methods provided herein such that the methods are performed concurrently or sequentially on the same solution/sample. Thus, for example, foam layer dissipation times may be measured for a particular test solution containing viable cells, and then one or more cell performance parameters may be assessed using that same test solution. However, it should be understood that viable cells are not needed to perform the indirect methods (e.g., measuring dissipation time or producing SEC chromatograms, as discussed herein).

A “shear-protectant additive,” as used herein, may refer to a compound that lowers the surface tension of a liquid. Examples of shear-protectant additives that may be used in accordance with the present disclosure include, without limitation, surfactants (e.g., nonionic surfactants), detergent, wetting agents, emulsifiers, foaming agents and dispersants. In some embodiments, the shear-protectant additive is a nonionic triblock copolymer, or poloxamer. A poloxamer is a nonionic triblock copolymer composed of a central hydrophobic chain of poly(propylene oxide) flanked by two hydrophilic chains of poly(ethylene oxide). In some embodiments, the poloxamer is a PLURONIC® block copolymer. Examples of PLURONIC® block copolymers include, without limitation, PLURONIC® F-68, PLURONIC® L-35, PLURONIC® F-127, PLURONIC® F-38 and PLURONIC® F-108. In some embodiments, the poloxamer is a KOLLIPHOR® block copolymer. In some embodiments, the poloxamer is a LUTROL® block copolymer. Additional examples of shear-protectant additives that may be used in accordance with the present disclosure include, without limitation, polyvinyl alcohol (PVA) and polyethylene glycol (PEG).

“Batch-to-batch variation” or “lot-to-lot variation,” used interchangeably herein, may refer to detectable differences in the effectiveness of samples of shear-protectant additives. For example, batch-to-batch variation of a shear-protectant additive may refer to differences among samples obtained from respective batches or lots of shear-protectant additives.

A shear-protectant additive may be added to a solution (e.g., comprising water or cell culture media) at a concentration of 0.01 g/L of solution to 10 g/L solution. For example, a shear-protectant additive may be added to a solution at a concentration of 0.01 g/L, 0.05 g/L. 0.1 g/L, 0.5 g/L, 1.0 g/L, 1.5 g/L, 2.0 g/L, 2.5 g/L, 3.0 g/L, 3.5 g/L, 4.0 g/L, 4.5 g/L, 5.0 g/L, 5.5 g/L, 6.0 g/L, 6.5 g/L, 7.0 g/L, 7.5 g/L, 8.0 g/L, 8.5 g/L, 9.0 g/L, 9.5 g/L or 10 g/L solution. In some embodiments, more than 10 g/L of shear-protectant additive may be added to the solution. In some embodiments, a shear-protectant additive may be added to a solution at a concentration of 1.2 g/L solution, 1.5 g/L solution or 1.8 g/L solution.

A solution, as provided herein may comprise one or more of a variety of liquid solvents. For example, in some embodiments, the solvent is water (e.g., purified water such as water for pharmaceutical use (WPU)), buffer (e.g., phosphate buffered saline), or cell culture media. Cell culture media for use in accordance with the present disclosure includes, without limitation, Dulbecco's Modified Eagle Medium (DMEM), Roswell Park Memorial Institute Medium (RPMI) and Minimal Essential Media (MEM). The cell culture media may be serum-free, or it may contain serum. In some embodiments, the cell culture media may contain additives such as, for example, interferons, cytokines, growth factors, amino acids, peptone, hydrolysate, peptides and/or other supplements that may regulate cell growth and/or proliferation. Other liquid solvents may be used in a solution in accordance with the present disclosure.

A “working volume” of solution (e.g., comprising water or cell culture media with or without cells), as used herein, may refer to the actual volume of solution used to perform an assay. In some embodiments, the working volume of the solution in a vessel (e.g., shake flask) may be 10% to 30% of the volume of the vessel. For example, a 1 L shake flask may contain a 100 ml working volume of solution. In some embodiments, the working volume of the solution in the vessel may be 10%, 15%, 20%, 25% or 30% of the total volume of the vessel. In some embodiments, the working volume may be less than 10% or more than 30% of the total volume of the vessel, which may depend on other conditions such as, for example, shake speed, orbit diameter and culture period. In some embodiments, the working volume may be a volume of solution in which, in combination with shake speed, orbit diameter and time, bubbles can be produced. In some embodiments, a vessel (e.g., shake flask) has a volume of 1 L and the working volume is 50 ml to 500 ml. In some embodiments, the vessel has a volume of 1 L and the working volume is 50 ml, 100 ml, 150 ml, 200 ml, 250 ml, 300 ml, 350 ml, 400 ml, 450 ml or 500 ml.

A “small-scale” method or system (e.g., cell culture method or system), as used herein, may refer to a method or system that uses vessels (e.g., shake flasks such as baffled shake flasks) with volumes of 10 L or less. For example, a small-scale system may refer to a system that uses vessels (e.g., shake flasks such as baffled shake flasks) with a volume of 125 mL, 500 mL, 1 L, 2 L, 2.5 L, 3 L, 5 L or 10 L. In some embodiments, a small-scale system may refer to a system that uses vessels with a volume of 125 mL to 3 L. By contrast, a “large-scale” method or system, as used herein, may refer to a method or system that uses vessel volumes of greater than 10 L. For example, a large-scale system may refer to a system that uses bioreactors (e.g., sparged bioreactors) with a volume of 20 L, 50 L, 100 L, 250 L, 500 L, 1000 L or 2000 L, or more. Other examples of small scale vessels include, without limitation, vials and test tubes. In some embodiments, baffled shake flasks are used, which provide for enhanced foam formation.

A “shake flask,” as used herein, refers to a small-scale vessel for holding solution (e.g., comprising water or liquid cell culture media), is suitable for shaking and permits aeration. A shake flask is “suitable for shaking” if most of the solution will remain in the flask when shaken in accordance with the methods of the present disclosure. In some embodiments, the shake flask is a baffled shake flask (e.g., an Erlenmeyer or conical flask) with, for example, a substantially flat bottom with any pattern of indentations extending inward (e.g., folds, ridges, protrusions and/or concentric rings), a conical body and a cylindrical neck. In some embodiments, the volume of the shake flask may be 125 mL to 10 L. For example, the volume of the shake flask may be 125 mL, 500 mL, 1 L, 2 L, 2.5 L, 3 L, 5 L or 10 L. In some embodiments, the volume of the shake flask (e.g., baffled shake flask) may be 125 mL to 3 L. The shake flask, in some embodiments, may be made of glass or plastic (e.g., polycarbonate, polypropylene, polystyrene, polyethylene, nylon, Teflon, polyvinyl chloride or polyethylene terephthalate).

To produce air bubbles and/or a foam layer in a solution (e.g., comprising water or cell culture media with or without cells), a vessel containing the solution may be agitated. Thus, air bubbles and/or a foam layer may be produced by shaking the solution (e.g., with an orbital shaker), using a stir bar (e.g., magnetic stir bar), vortexing, sparging, or by other means of agitation. In some embodiments, a solution is shaken, for example, in a shake flask. In some embodiments, the solution is shaken with a shaking apparatus such as, for example, an orbital shaker. The orbital diameter of the shaker, in some embodiments, may be 10 mm to 50 mm. For example, the orbital diameter of the shaker may be 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mmm or 100 mm.

The speed at which the solution (e.g., with or without cells) is shaken may be 100 revolutions per minute (rpm) to 300 rpm. For example, the solution may be shaken, e.g., in a shake flask, at a speed of 100 rpm, 150 rpm, 200 rpm, 250 rpm or 300 rpm, or more.

It should be appreciated that other techniques may be used for generating bubbles (e.g., to produce foam/a layer of foam).

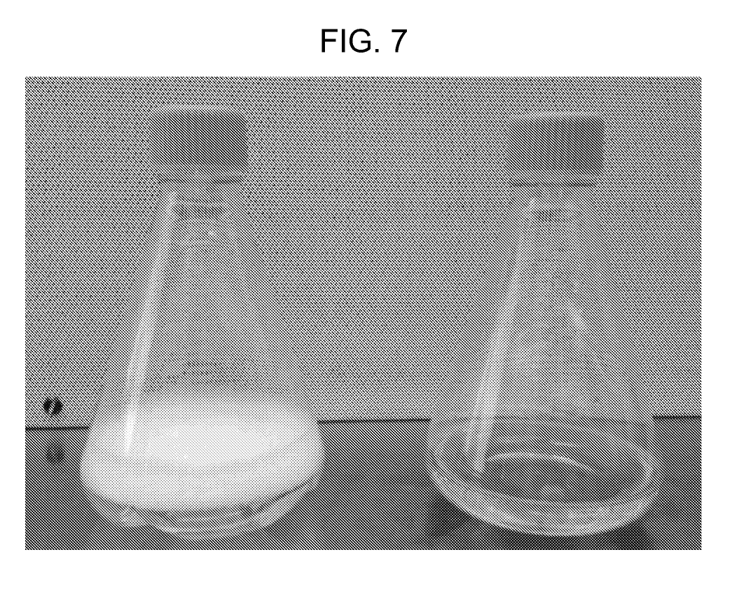

A “foam layer” of a solution, as used herein, refers to a layer of bubbles that substantially covers the surface area of a bulk liquid layer of the solution in a vessel. Accordingly, a “bulk liquid layer,” as used herein, refers to the liquid portion of a solution that does not contain a foam layer. For example, the photograph on the left in FIG. 7 shows a baffled shake flask containing a solution with a shear-protectant additive that has been shaken for a period of time sufficient to produce a foam layer which sits on top of the liquid bulk layer. A period of time sufficient to produce such a foam layer can depend on several factors including, inter alia, the type of vessel in which the solution resides, the type of method used to introduce air bubbles into the solution to form the foam layer, and the components of the solution.

Examples of components that affect foam formation include, without limitation, the type of shear-protectant additive, antifoaming agents (e.g., antifoam Q7-2587), and other hydrophobic agents present in the solution.

In some embodiments, a period of time sufficient to produce a foam layer will be a period of time sufficient to produce a foam layer that is 10% to 300%, or more, of the total volume of the bulk liquid layer. For example, the volume of the foam layer may be 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, or more, of the total volume of the bulk liquid layer. In some embodiments, the ratio of the volume of the foam layer to the volume of the bulk liquid layer is about 1:1, or greater than 1:1. In some embodiments, the ratio of the volume of the foam layer to the volume of the bulk liquid layer is 2:1, 3:1, 4:1 or 5:1.

In some embodiments, the minimum volume of the foam layer necessary to assess the effectiveness of a shear-protectant additive for protecting cells from shear damage is a volume sufficient to cover the top of the bulk liquid layer. Generally, if a foam layer is visible, it may be sufficient for use is assessing the effectiveness of the additive. In some embodiments, the layer of foam is 1 mm thick to 100 mm thick. For example, the thickness of the layer of foam may be 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, or more.

A period of time sufficient to produce a foam layer may be 5 minutes to 48 hours, or more. For example, a solution may be agitated (e.g., shaken) for 5 minutes, 10 minutes, 15 minutes, 20 minutes, 30 minutes, 45 minutes, 60 minutes, 2 hours, 8 hours, 12 hours, 36 hours or 48 hours, or more. In some embodiments, a period of time sufficient to produce a foam layer may be less than 5 minutes, for example, 4, 3, 2, or 1 minute. In some embodiments, a period of time sufficient to produce a foam layer may be 15 minutes to 12 hours.

Some aspects of the present disclosure provide methods of directly assessing cell viability. Thus, in some embodiments, a solution containing viable cells may be agitated (e.g., shaken) for a period of time to produce bubbles in the solution in an amount sufficient to cause a greater than 5% drop in cell viability compared to the initial cell viability. In some embodiments, the cells may be agitated for a period of time to produce bubbles in the solution in an amount sufficient to cause a greater than 10% or greater than 15% drop in cell viability compared to the initial cell viability. In some embodiments, the solution may be agitated for a period of time to produce bubbles in the cell culture media in an amount sufficient to cause 5% to 25% drop (e.g., 5%-10%, 10%-15%, 15%-20%, 20%-25%, 10%-20%) in cell viability compared to the initial cell viability. This period of time (culture period) may depend on other conditions such as, for example, the cell type, the working volume of solution, the orbital diameter of the shaker, and/or the shake speed. The “initial cell viability,” as used herein, may refer to the viability of the cells before culturing/incubating the cells (e.g., culture period=zero) under test conditions (e.g., with bubbles). In some embodiments, initial cell viability is obtained from cells in a solution (e.g., cell culture media) that contains 0.02-0.2 g/L shear-protectant additive but does not contain a layer of foam/bubbles. In some embodiments, initial cell viability is obtained from cells in a solution that contains 0.02-5.0 g/L (e.g., 1.0, 2.0, 3.0, 4.0, 5.0 g/L) shear-protectant additive but does not contain a layer of foam/bubbles.

“Cell viability” herein refers to a measure of the number of cells that are viable (e.g., alive and capable of growth). Assays for determining cell viability are well-known in the art and include, for example, an ATP test, calcein AM staining, a clonogenic assay, an ethidium homodimer assay, Evans blue staining, fluorescein diacetate hydrolysis/Propidium iodide staining (FDA/PI staining), flow cytometry, formazan-based assays (MTT/XTT), green fluorescent protein reporter assay, LDH reporter assay, methyl violet staining, propidium iodide staining, and DNA stains that can differentiate necrotic, apoptotic and normal cells (Lecoeur H, Experimental Cell Research, 277(1): 1-14, 2002), resazurin staining, Trypan Blue staining, a living-cell exclusion dye (dye only crosses cell membranes of dead cells), and a TUNEL assay. In some embodiments, cell viability may be measured by determining the total cell count minus the count of nonviable or dead cells. Other viable cell assays may also be used. In some embodiments, cell viability may be determined using a commercially-available automated cell culture analysis system (e.g., Cedex HiRes Analyzer, Roche Applied Science, IN).

“Viable cell density” herein refers to the number of viable cells per unit volume of solution (e.g., cell culture media). Assays for determining viable cell density are well-known in the art, any of which may be used in accordance with the present disclosure. In some embodiments, viable cell density may be determined using a commercially-available automated cell culture analysis system (e.g., Cedex HiRes Analyzer, Roche Applied Science, IN). “Normalized viable cell density” is the viable cell density divided by initial viable cell density.

“Cell performance parameters” herein refers to any parameter than can be measured that is indicative of cell viability and/or cell growth and/or cell metabolism. Examples of cell performance parameters include, without limitation, cell viability, viable cell density, protein titer, lactate dehydrogenase (LDH) in spent media, pH, metabolite production and carbohydrate consumption.

In some aspects, methods comprise culturing cells, while in other aspects, methods do not include culturing cells.

In some embodiments, cells may be cultured at a temperature of 30° C. to 40° C. For example, the temperature at which cells are cultured may be 30° C., 31° C., 32° C., 33° C., 34° C., 35° C., 36° C., 37° C., 38° C., 39° C. or 40° C. In some embodiments, cells are cultured at a temperature of 35° C. In some embodiments, cells may be cultured at room temperature. In some embodiments, cells may be cultured in an environment that is not controlled for temperature.

In some embodiments, cells may be cultured in the presence of CO₂, for example, in a CO₂ incubator. In some embodiments, cells may be cultured at 3% CO₂ to 10% CO₂. For example, the cells may be cultured at 3% CO₂, 4% CO₂, 5% CO₂, 6% CO₂, 7% CO₂, 8% CO₂, 9% CO₂ or 10% CO₂. In some embodiments, cells may be cultured at 5% CO₂. In some embodiments, cells may be cultured at 0% CO₂. In some embodiments, cells may be cultured in an environment that is not controlled for CO₂.

Any cell type may be used in accordance with the present disclosure. In some embodiments, mammalian cells are used. In some embodiments, non-mammalian cells are used. In other embodiments, bacterial cells, insect cells, microalgae cells, fungal cells (including yeast cells) or plant cells may be used. Examples of cells that may be used herein include, without limitation, 293-T, 3T3, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CHO (e.g., CHO-K1, CHO-DXB11 (also referred to as CHO-DUKX), CHO-pro3, CHO-DG44 and CHO-S), CML T1, CMT, COR-L23, COR-L23/5010, COR-L23/CPR, COR-L23/R23, COS-7, COV-434, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, High Five, HL-60, HMEC, HT-29, HUVEC, J558L, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NSO, NW-145, OPCN/OPCT, Peer, PNT-1A/PNT 2, Raji, RBL cells, RenCa, RIN-5F, RMA/RMAS, Saos-2 cells, Sf21, Sf9, SiHa, SKBR3, SKOV-3, SP 2/0, T-47D, T2, T84, THP1, U373, U87, U937, VCaP, Vero, WM39, WT-49, X63, YAC-1 and YAR cells.

Reference values as provided herein may be based on a positive control used in the method of the present disclosure.

A “reference value,” as used herein, may refer to a value that is characteristic of a shear-protectant additive that is suitable for large-scale system (e.g., using a large-scale sparged bioreactor). In some embodiments, a shear-protectant additive is “suitable” for large-scale systems (e.g., large-scale cell culture) if its use results in a drop in viability of less than or 20%, or less than or 10%, or less than or 5%. In some embodiments, a reference value may be based on foam layer dissipation time of a shear-protective additive known to be effective for protecting cells against shear damage. In some embodiments, a reference value may be based on high molecular weight peaks of a foam layer sample of a shear-protective additive known to be effective for protecting cells against shear damage. In some embodiments, a reference value may be based on the hydrophilic-lipophilic balance (HLB) value of sample of a shear-protective additive known to be effective for protecting cells against shear damage. In other embodiments, a reference value may be based on one or more cell performance parameters of cells cultured under the same conditions as the cells being measured in accordance with the present disclosure, with the exception that cells on which the reference value is based are cultured in the presence of a shear-protectant additive (or a batch of shear-protectant additive) known to be effective (or suitable) for protecting cells from shear damage. In some embodiments, a reference value may be a value that is characteristic of an unsuitable composition. For example, a composition of interest may be compared to a suitable reference to determine whether it is different from the suitable reference, or to an unsuitable reference to determine it is the same or similar to the unsuitable reference.

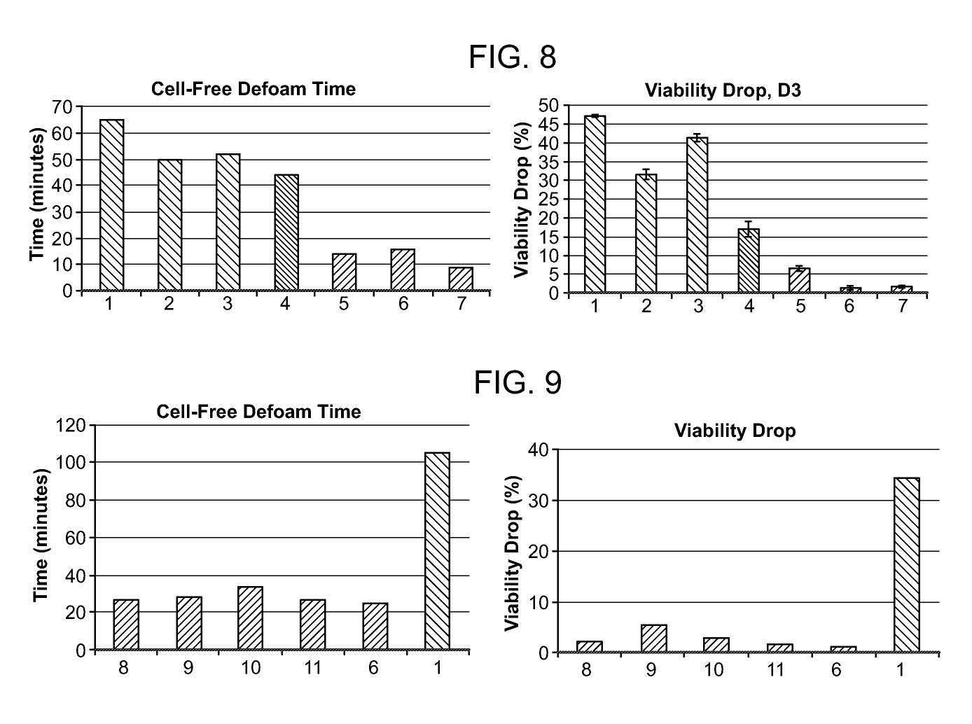

In some embodiments, a reference value may be “pre-determined.” That is, the reference value may be obtained, prior to the assay being performed on the test sample, from one or more control samples such as, for example, one or more samples of the same type of shear-protectant additive obtained from a lot known to be effective for protecting cells from shear damage (e.g., each sample may be from different lots of PLURONIC® and/or KOLLIPHOR®). FIGS. 8-10 include examples of pre-determined reference values for small-scale (e.g., cell-free) methods provided herein, such as those that use shake flasks (e.g., baffled shake flasks) having a volume of less than 10 L. In some embodiments, a reference value is 40 minutes, 35 minutes, 30 minutes, 25 minutes, 20 minutes, 15 minutes, or 10 minutes.

Some assays provided herein can be used to directly assess the effectiveness of a sample of shear-protectant additive on protecting cells from shear damage. Direct methods include viable cells in solution, whereby, in some embodiments, the viability of the cells is directly assessed in the presence of a sample of a shear-protective additive. Based on that assessment, a shear-protectant additive is selected for further use.

In some embodiments, a shear-protectant additive may be selected if the viability of cells cultured in accordance with the present disclosure drops by (decreases by) less than 10% as compared to the initial cell viability. In some embodiments, a shear-protectant additive may be selected if the viability of cells cultured in accordance with the present disclosure drops by less than 9%, less than 8%, less than 7%, less than 6%, or less than 5% as compared to the initial cell viability.

In some embodiments, a shear-protectant additive may be selected if cells cultured/grown in accordance with the present disclosure have a cell viability of greater than 80%. In some embodiments, a shear-protectant additive may be selected if the cultured in accordance with the invention have a cell viability of greater than 85%, greater than 90%, greater than 95% or greater than 98%. In some embodiments, a shear-protectant additive may be selected if cells cultured in accordance with the present disclosure have a cell viability of 80% to 99%.

In some embodiments, a shear-protectant additive may be selected if the cells cultured in accordance with the present disclosure have a viable cell density comparable to the viable cell density of cells cultured, under similar conditions, in the presence of a shear-protectant additive known to be effective for protecting cells from shear damage.

In some embodiments, a shear-protectant additive may be selected if the cells cultured in accordance with the present disclosure have a viable cell density of greater than 12e6 vc/mL. In some embodiments, a shear-protectant additive may be selected if the cells cultured in accordance with the present disclosure have a viable cell density of greater than 13e6 vc/mL, greater than 14e6 vc/mL, greater than 15e6 vc/mL, or greater than 16e6 vc/mL cell culture media. In some embodiments, a shear-protectant additive may be selected if the cells cultured in accordance with the present disclosure have a viable cell density of 12e6 vc/mL to 16e6 vc/mL (e.g., 12e6-13e6 vc/mL, 12e6-14e6 vc/mL, 12e6-15e6 vc/mL, 14e6-15e6 vc/ml).

In some embodiments, a shear-protectant additive may be selected if cells cultured in accordance with the present disclosure have a protein titer of greater than 30 mg/L of cell culture media. In some embodiments, a shear-protectant additive may be selected if cells cultured in accordance with the present disclosure have a protein titer of greater than 40 mg/L, greater than 50 mg/L, or greater than 60 mg/L of cell culture media. In some embodiments, a shear-protectant additive may be selected if cells cultured in accordance with the present disclosure have a protein titer of 30 mg/L to 60 mg/L (e.g., 30-40 mg/L, 40-50 mg/L, 50-60 mg/L, 40-50 mg/L). Protein titer herein refers to the concentration of the product protein in solution (e.g., cell culture media). Assays for determining protein titer are well-known in the art, any of which may be used in accordance with the present disclosure. In some embodiments, protein titer may be determined using high-performance liquid chromatography (HPLC) (e.g., Taqman, Applied Biosystems, Agilent Technologies, CA).

The reference values for cell viability, viable cell density and cell titer may be determined or provided independent of the method of the present disclosure. Thus, the reference value may be a predetermined reference value. For example, the reference value for cell viability may be 80%, 85%, 90%, 95% or 98%. In some embodiments, the reference value for cell viability may be greater than or 80%, greater than or 85%, greater than or 90%, greater than or 95%, or greater than or 98%. As other examples, the reference value for viable cell density may be 12e6 viable cells/milliliter (vc/mL), 13e6 vc/ml, 14e6 vc/mL, 15e6 vc/mL, or 16e6 vc/mL. In some embodiments, the reference value for viable cell density may be greater than or 12e6 vc/mL, greater than or 13e6 vc/ml, greater than or 14e6 vc/mL, greater than or 15e6 vc/mL, or greater than or 16e6 vc/mL. As yet other examples, the reference value for protein titer may be greater than or 30 mg/L, greater than or 40 mg/L, greater than or 50 mg/L, or greater than or 60 mg/L in cell culture media. In some embodiments, a reference value may refer to a value measured before the cells are cultured under test conditions (e.g., culture period=zero).

Other assays provided herein can be used to indirectly assess the effectiveness of a sample of shear-protectant additive on protecting cells from shear damage. Such indirect methods, in some embodiments, are cell-free and thus do not directly assess cell. Rather, such indirect methods, based on the results of the assay, permit a correlation to be made with respect to the effectiveness of the shear-protective additive. Based on that correlation, a shear-protectant additive is selected for further use.

Methods and compositions provided herein may be used to evaluate a shear-protectant composition to determine whether it is suitable for use in a cell growth and/or protein production procedure (e.g., whether the composition sufficiently protects cells from shear damage). In some embodiments, a lot of a shear-protectant composition that has at least one property that is characteristic of an unsuitable shear-protectant composition is not selected for further use, for example, in a cell growth and/or protein production procedure. For example, a shear-protectant composition may be evaluated to determine whether it contains highly hydrophobic components that are (a) different from (e.g., statistically higher than) an amount characteristic of a known suitable shear-protectant composition, and/or (b) similar to (e.g., statistically significantly similar to) an amount characteristic of a known unsuitable shear-protectant composition. In some embodiments, the hydrophobicity of a shear-protectant composition may be assessed using reverse phase high performance liquid chromatography (RP-HPLC). Thus, in some embodiments, a test sample of a shear-protectant composition may be evaluated by RP-HPLC to determine whether it has a chromatographic profile similar to that of a shear-protectant composition known to be unsuitable for use in, for example, cell culture, in which case the shear-protectant composition from which the test sample was obtained is not selected for further use. Likewise, a test sample of a shear-protectant composition may be evaluated by RP-HPLC to determine whether it has a chromatographic profile similar to that of a shear-protectant composition known to be suitable for use in, for example, cell culture, in which case the shear-protectant composition from which the test sample was obtained is selected for further use. Other assays known in the art (including for example, but not limited to, other chromatographic techniques) may also be used to assess the hydrophobicity and/or molecular weight profile of a shear-protectant composition. In some embodiments, the hydrophobicity of one or more fractions (e.g., one or more fractions having different molecular weight ranges) is evaluated. In some embodiments, one or more of the properties described herein is evaluated for a composition of interest and compared to the same property of a known suitable or unsuitable composition. In some embodiments, if the property is similar (e.g., with statistical significance) to that of a suitable composition and/or different (e.g., with statistical significance) from that of an unsuitable composition, then the composition (e.g., a poloxamer lot or batch) may be used for cell growth and/or protein production. In contrast, if the property is different (e.g., with statistical significance) from that of a suitable composition and/or similar (e.g., with statistical significance) to that of an unsuitable composition, then the composition (e.g., a poloxamer or batch) may be excluded from use in cell growth and/or protein production.

In some embodiments, methods and compositions provided herein are used to assess different lots of poloxamer 188. Poloxamer 188 (also referred to as PLURONIC® F-68, KOLLIPHOR® P-188, LUTROL® F-68). Poloxamer 188 has a hydrophilic-lipophilic balance (HLB) value of 29. The hydrophilic-lipophilic balance of a surfactant is a measure of the degree to which it is hydrophilic or lipophilic, determined by calculating values for the different regions of the molecule (see, e.g., Griffin W. C., Journal of the Society of Cosmetic Chemists 1 (5): 311-26; Griffin W. C., Journal of the Society of Cosmetic Chemists 5 (4): 249-56, each of which is incorporated by reference herein). As shown in Example 9 below, the addition to poloxamer 188 of even a small amount of a highly hydrophobic component can render poloxamer 188 unsuitable for use in, for example, cell growth and/or protein production procedure. Thus, in some embodiments, a lot or batch of poloxamer 188 that has a HLB value of less than 29 (e.g., less than 28, less than 27, less than 26, less than 25) is considered an unsuitable shear-protectant composition and is not selected for further use, for example, in a cell growth and/or protein production procedure.

In some embodiments, a shear-protectant composition may be evaluated to determine whether it contains high molecular weight components in an amount that is (a) different from (e.g., statistically higher than) an amount characteristic of a known suitable shear-protectant composition, and/or (b) similar (e.g., statistically significantly similar) to an amount characteristic of a known unsuitable shear-protectant composition. In some embodiments, the molecular weight of a shear-protectant composition may be assessed using size exclusion chromatography (SEC). Thus, in some embodiments, a test sample of a shear-protectant composition may be evaluated by SEC to determine whether it has a chromatographic profile similar to that of a shear-protectant composition known to be unsuitable for use in, for example, cell culture, in which case the shear-protectant composition from which the test sample was obtained is not selected for further use. Likewise, a test sample of a shear-protectant composition may be evaluated by SEC to determine whether it has a chromatographic profile similar to that of a shear-protectant composition known to be suitable for use in, for example, cell culture, in which case the shear-protectant composition from which the test sample was obtained is selected for further use. Other assays known in the art (e.g., including, but not limited to, mass spectrometry, other size based chromatography or separation techniques) may also be used to assess the molecular weight profile of a shear-protectant composition.

As discussed above, in some embodiments, methods and compositions provided herein are used to assess different lots of poloxamer 188. Poloxamer 188 has an average molecular weight of 8400 Daltons. Studies provided herein demonstrate that certain lots of poloxamer 188, for example, those that contain components having a molecular weight of greater than 12,000 Daltons (Da) (e.g., greater than 13,000 Da, greater than 14,000 Da), wherein these components are present in an amount that is greater (e.g., with statistical significance) that an amount of material of similar size (if present) in a poloxamer composition known to be suitable for cell growth and/or protein production, are considered unsuitable for use, for example, in a cell growth and/or protein production procedure. Thus, in some embodiments, a lot of poloxamer 188 that contains components having a molecular weight of greater than 12,000 Da is considered an unsuitable shear-protectant composition and is not selected for further use, for example, in a cell growth and/or protein production procedure if the amount of components having a molecular weight of greater than 12,000 is statistically higher than the amount of components having a molecular weight of greater than 12,000 in a known suitable poloxamer composition (or is statistically similar to an amount of components having a molecular weight of greater than 12,000 in a known unsuitable poloxamer composition).

A shear-protectant can be evaluated in any form that can be analyzed, for example, in the form of a powder, a solution, or any other form that can be analyzed to determine the presence of one or more properties that are characteristic of an unsuitable shear-protectant (e.g., components that are highly hydrophobic and/or have a high molecular weight).

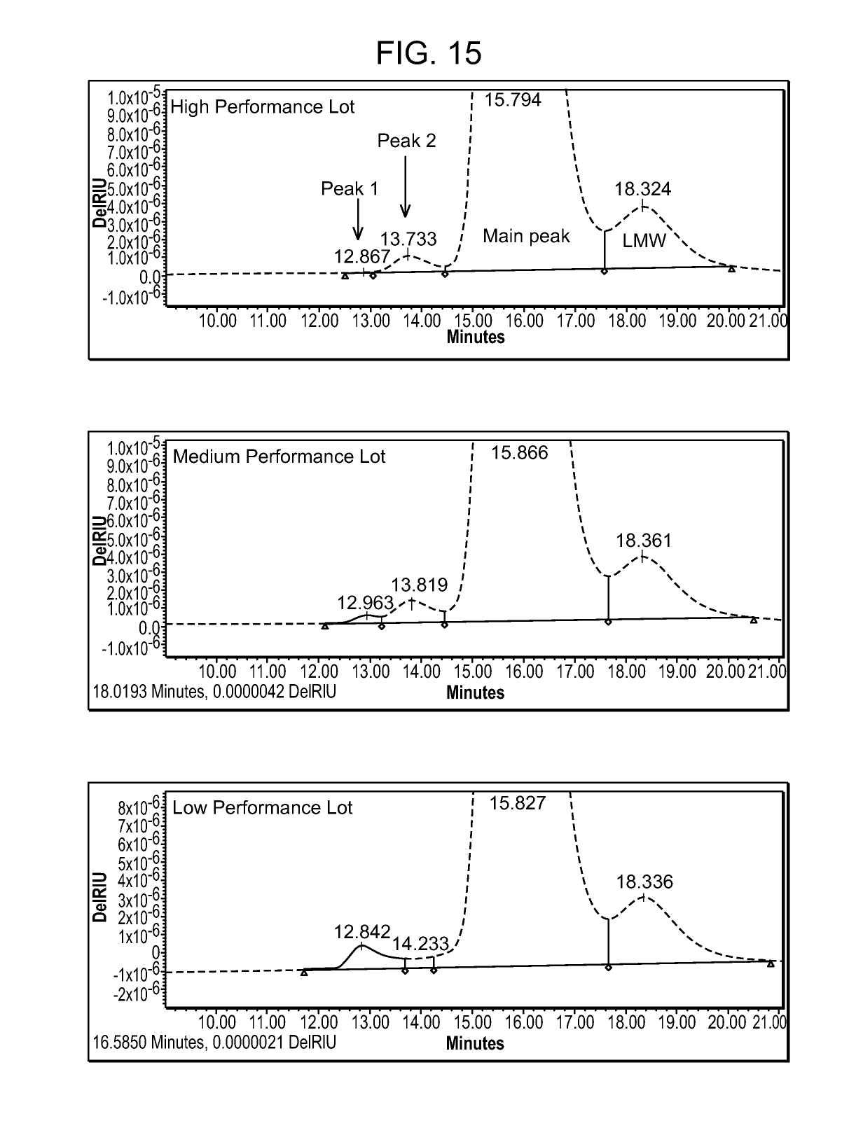

It should be appreciated that polymeric shear-protectant compositions can comprise a distribution of different polymers (e.g., having different sizes and/or relative content of the polymer components). In some embodiments, a polymeric shear-protectant composition is evaluated to determine whether it contains a distribution of polymers that is similar to (a) a composition that is known to be suitable for cell growth and/or protein production (e.g., on a large scale, for example in a manufacturing scale fermenter), and/or (b) a composition that is known to be unsuitable for cell growth and/or protein production. For example, FIG. 15 shows an SEC chromatographic comparison of the molecular weight profile of three different lots of poloxamer 188—a suitable (high performance) lot, an intermediate (medium performance) lot, and an unsuitable (low performance) lot. Such chromatograms may be used, for example, to assess additional lots of poloxamer 188, provided the SEC conditions are similar.

In some embodiments, the hydrophobicity of a shear-protectant composition is evaluated (e.g., measured or determined) without fractionating the composition and/or without isolating certain components from the composition. However, in some embodiments, the hydrophobicity of one or more fractions (e.g., one or more size ranges of components of the poloxamer composition, or one or more peaks of the poloxamer composition, for example when fractionated using size fractionation, e.g., SEC) of the shear-protectant composition is evaluated. For example, in some embodiments one or more fractions having different molecular weight ranges are evaluated. In some embodiments, a foam layer produced shaking or otherwise agitating or mixing a shear-protectant composition is evaluated. The foam layer produced by agitation of a solution containing a shear-protectant additive is enriched in hydrophobic components. Fractionation of this this layer, or a sample of this layer, obtained from a composition containing an unsuitable shear-protectant, shows that the highly hydrophobic foam layer also contains high molecular weight components (e.g., in an amount greater than found in foam derived from suitable shear-protectant). Thus, in some embodiments, a method of the present disclosure includes producing a foam layer in a composition containing a sample of a shear-protectant, collecting the foam layer (e.g., after removing the bulk layer and allowing the foam to dissipate), and evaluating the molecular weight of the components of the foam layer. The shear-protectant may then be selected for further use if the molecular weight (and/or the relative amounts) of the components of the foam layer is comparable to the molecular weight (and/or the relative amounts) of the components of the foam layer of a shear-protectant known to be suitable. Conversely, the shear-protectant may not be selected for further use if the molecular weight (and/or the relative amounts) of the components of the foam layer is comparable to the molecular weight (and/or the relative amounts) of the components of the foam layer of a shear-protectant known to be unsuitable.

In some embodiments, the molecular weight profile of a shear-protectant composition is evaluated (e.g., measure or determined). In some embodiments, the relative amount of one or more high molecular weight components present in a shear-protectant composition can be evaluated by determining the relative amount of one or more high molecular weight fractions in the composition. In some embodiments, the relative amount of high molecular weight components in a shear-protectant composition being evaluated is determined relative to a suitable reference (e.g., the total amount of material in the composition, the amount of material having an average molecular weight of the composition, the amount of one or more lower molecular weight fractions of the composition, or other suitable reference). In some embodiments, the amount of shear-protectant material in one or more high molecular weight fractions (e.g., the highest 5%, 10%, 15%, 20%, 25%, 30%, or 35% of the molecular weight range of the shear-protectant composition being evaluated) is determined and compared to (e.g., divided by) a suitable reference amount of material for the composition being evaluated. However, other calculations may be used to determine whether the molecular weight profile of a shear-protectant composition is similar to or different from that of a suitable or unsuitable shear-protectant composition that is being used as a reference profile.

In some embodiments, a shear-protectant composition is identified as suspicious if it contains an amount of high molecular weight material that is higher (e.g., statistically higher) than a suitable composition. In some embodiments, the high molecular weight material is identified as a particular peak in a molecular weight profile. In some embodiments, the high molecular weight material is identified as one or more peaks above a particular reference molecular weight. However, in some embodiments, the presence of a suspicious amount of a high molecular weight material can result in a change in the overall distribution (e.g., the presence of a shoulder or bump in the higher molecular weight fractions of the molecular weight distribution of a composition being evaluated indicating the presence of a higher than expected amount of high molecular weight material even if one or more discrete peaks are not identified).

Assessing the effectiveness of a shear-protectant additive (or a particular lot of a shear-protectant additive) in an indirect assay can, in some embodiments, include measuring the duration of time during which the foam layer of a solution dissipates (or substantially liquefies or substantially disappears). This period of time is referred to herein as “dissipation time.” Dissipation time may refer to a period of time that encompasses the total time measure between when a solution is no longer agitated (e.g., no longer shaking, is in a steady state) and the time that substantially all foam in the foam layer liquefies (e.g., the foam layer is no longer visible or separate from the bulk layer). Dissipation time may also refer to intermediate periods of time between when a shake flask is no longer shaking to the time when a proportion of the foam liquefies (e.g., ¾, ½, ¼ volume of the foam liquefies, or 10%, 20%, 30%, 40%, 50%, 60%, 70%, 80%, 90% or 95% of the foam layer liquefies). The dissipation time of a test sample of a shear-protectant additive (e.g., an additive suspected of contamination, or a “suspicious” lot) may, in some embodiments, be compared to the dissipation time of a control sample of a shear-protectant additive or to a reference value. The control sample may be one or more samples of the same type of shear-protectant additive, for example, obtained from a lot known to be effective for protecting cells from shear damage (e.g., a suitable lot). In some instances, the control sample and the test sample are both used in an assay. In some embodiments, a reference value may be “pre-determined.” Based on a comparison to reference values based on control samples, a determination may be made with regard to whether a suspicious sample is a suitable sample or an unsuitable sample. Typically, suitable samples are selected for further use, for example, in a cell culture assay.

In some embodiments, an antifoaming agent may be added to a solution to reduce the amount of foam generated, which can, in turn, reduce the dissipation time, thereby shortening the time of the assay. In some instances, when added to a test sample and a control sample, antifoaming agent can better resolve differences between a test sample and a control sample. For example, the difference between dissipation times of a test sample and a control sample may be greater with the inclusion of an antifoaming agent. The antifoaming agent may be silicone-based, oil-based or water-based. Examples of antifoaming agents that may be used in accordance with the present disclosure include, without limitation, Andifoam DF, Pluriol® P 1000, Pluriol® P 2000, Pluriol® P 4000, BYK® A 501, BYK® A 515, BYK® A 550, BYK® A 555, Entschaumer L, Silcolapse® 426R, Kemamide® W-40 DF, Foamaster® 8034E, Xiameter® PMX-200 10,000 cSt, Xiameter® PMX-200 12,500 cSt, Xiameter® PMX-200 30,000 cSt, Xiameter® PMX-200 5,000 cSt, Xiameter® PMX-200 60,000 cSt, Mark® I 489, Solulub 144, Hallco® C-451, Dumacil 100, Dumacil 402, Dumacil 402-FG, Dumacil 402-FG-K, Antischiuma FL3, Inovol AF12, Antitack BTO-7, KP 1300, Baysilone Antifoam TP 3757, Baysilone Antifoam TP3861, Baysilone® Antifoam 3099, Aluminium stearate, Addovate® DD 1092, Lial® 123A, Lial® 125A, Lial® 145 A, 2-EH, Antifoam SAF-105, Antifoam SAF-110, Antifoam SAF-119FG, Antifoam SAF-120, Antifoam SAF-121, Amgard TBEP, Colloid™ 581B, Colloid™ 635, Colloid™ 675, Colloid™ 681F, Struksilon 8304, Struksilon 8314, T-SIL 10000, Octosperse TS-10, Octosperse TS-30, HDK® H2000, Wacker® AK 100 Silicone Fluid, Wacker® AK 1000 Silicone Fluid, Wacker® AK 12500 Silicone Fluid and Wacker® AK 35 Silicone Fluid.

In some embodiments, a test sample of shear-protectant additive may be selected, for example, for further use in a cell culture assay. A sample may be selected if its dissipation time is comparable to a control sample, or reference value, as discussed above. In some embodiments, a sample of a shear-protectant additive is selected if its foam layer dissipation time is less than the control sample or the reference value. Such comparisons and selections can be made using, for example, standard statistical analyses and techniques.

Other aspects of the disclosure provide for methods of (a) producing a foam layer in a test solution that comprises a sample of shear-protectant additive at a concentration of 0.01 g/L to 10 g/L test solution, (b) collecting a liquefied foam layer sample from the test solution, (c) producing a size exclusion chromatography (SEC) chromatogram of the liquefied foam layer sample, (d) comparing the high molecular weight peak of the SEC chromatogram to a reference value, and (e) selecting the shear-protectant additive if the high molecular weight peak of the SEC chromatogram is comparable to the reference value. In some embodiments, the reference value is a pre-determined value. In some embodiments, the reference value based on a high molecular weight peak of a SEC chromatogram from (e.g., obtained from) a control sample of a solution containing a sample of a shear-protectant additive known to be effective for protecting cells against shear damage. In some embodiments, the control sample is from the bulk layer of the test solution. In some embodiments, the test solution is a cell-free solution.

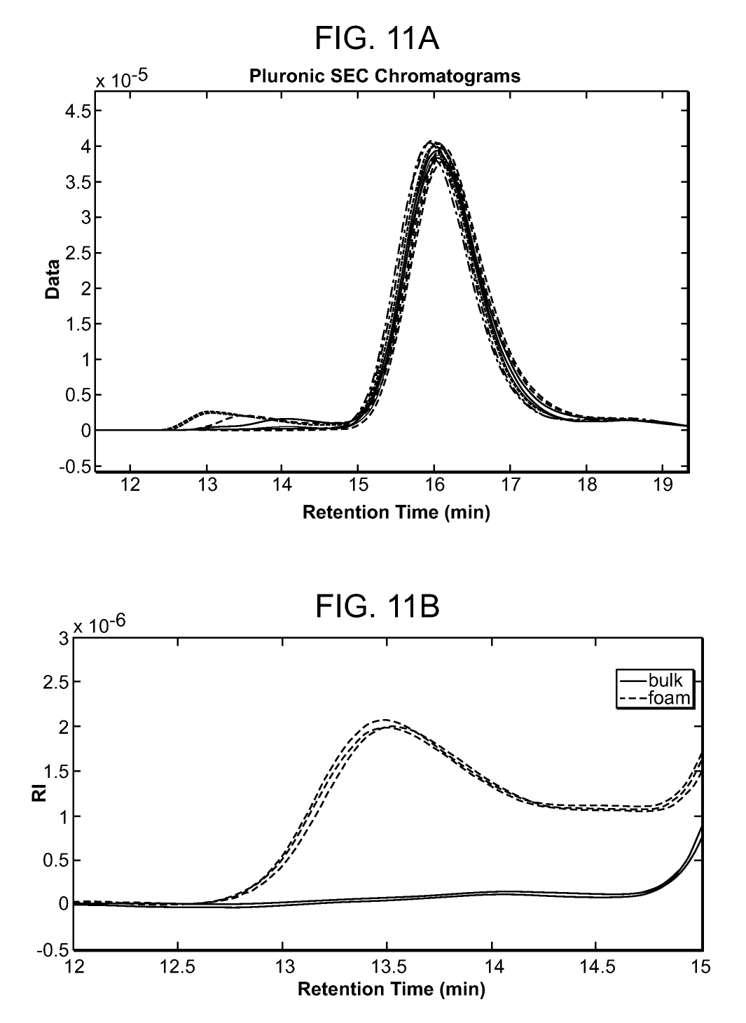

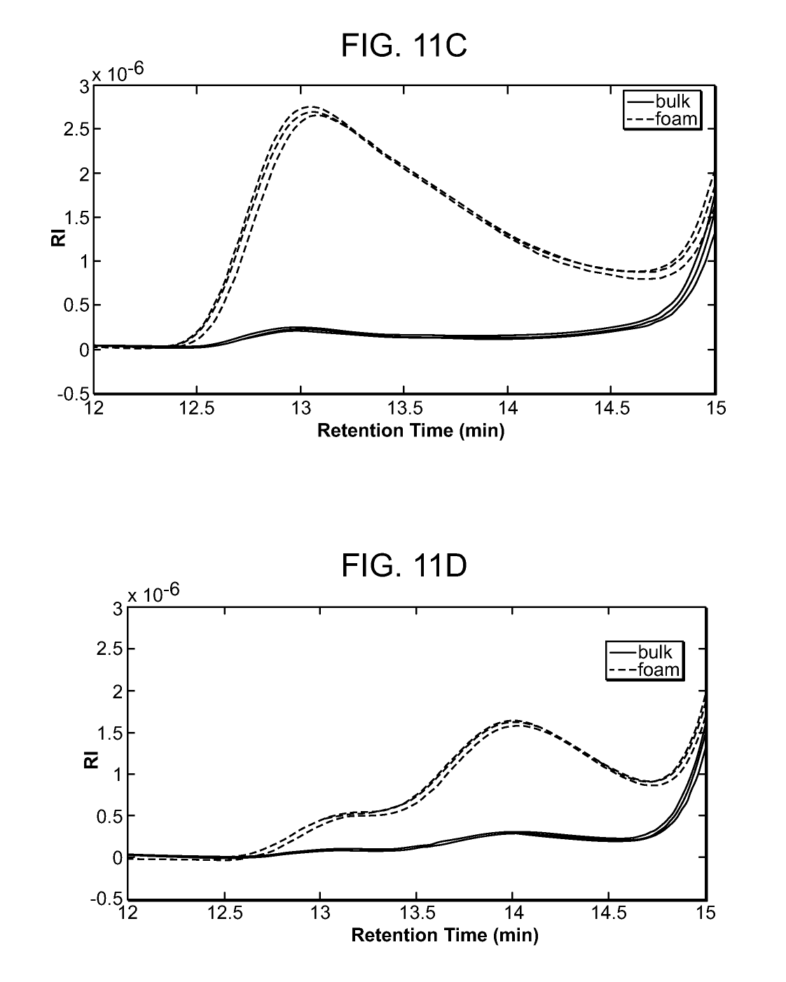

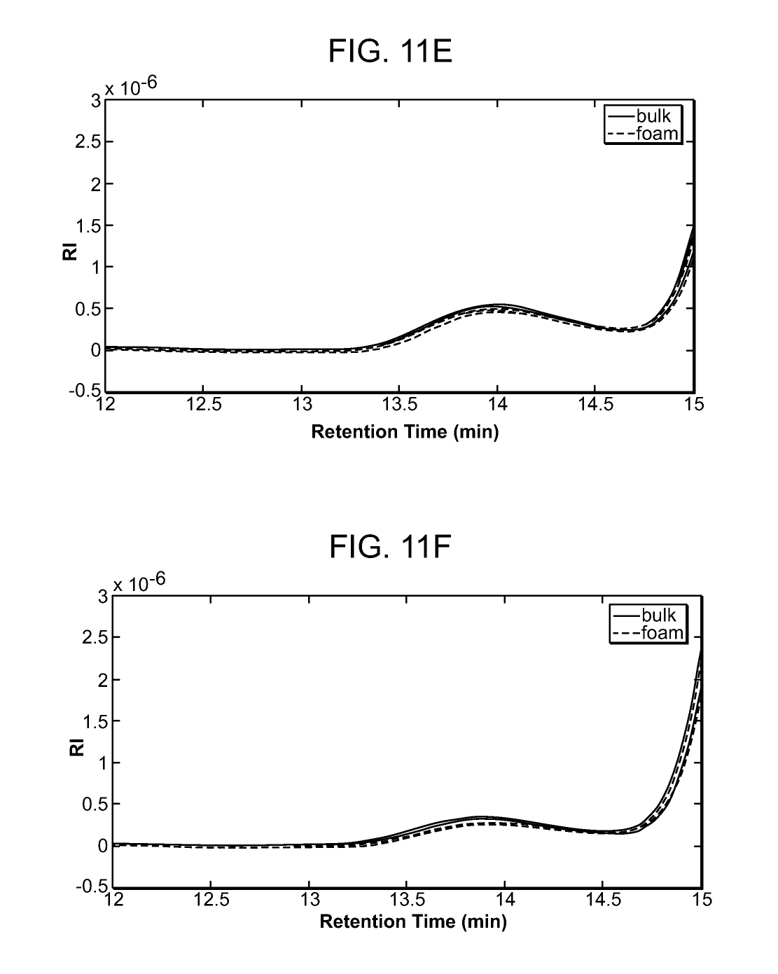

Size-exclusion chromatography (SEC) is a chromatographic method in which molecules in solution are separated by their size, and in some cases, molecular weight (Paul-dauphin et al. Energy & Fuels. 6 21 (6): 3484-3489). In some embodiments, the methods herein provide for the selection of test samples of shear-protectant additives based on a SEC chromatogram profile. The first peak of a chromatogram, representative of high molecular weight portions (large molecule) of a foam layer of sample, differs among suitable and unsuitable samples of shear-protectant additives. Such a chromatogram may be produced, as follows: the bulk layer of a solution is collected, leaving the foam layer to liquefy. The liquefied foam layer is then collected. A sample of each of the bulk layer and the liquefied foam layer is subjected to SEC to produce a chromatogram. The first peak of the chromatogram is representative of molecules larger than a select pore size of a SEC filter. FIG. 11E is representative of a chromatogram of a suitable sample of PLURONIC® F-68, showing that there is little difference between the first peak produced using the liquefied foam layer (bottom three lines, n=3) and first peak produced using the bulk layer (top three lines) (Retention Time=14 min). By contrast, 11B is representative of a chromatogram of an unsuitable sample of PLURONIC® F-68, showing that there is a large difference between the first peak of the liquefied foam layer (bottom three line, n=3), representative of large molecules present in the sample, and the first peak of the bulk layer (top three lines) (Retention Time=13.5 min). Thus, the refractive index (RI) of the first peak of the liquefied foam layer of an unsuitable test sample (or a sample that is less effective in protecting cells relative to a control sample) is greater than the RI of the first peak of the bulk layer of that same test sample. A chromatogram for an “intermediate” sample is show in FIG. 11D. The difference in height (and area) between the first peaks of the liquefied foam layer and the bulk layer is not as great as the difference observed in a chromatogram from an unsuitable sample (e.g., shown in FIG. 11B).

Methods provided herein are particularly useful for selecting shear-protectant additives that may be used in large-scale manufacturing processes (e.g., large-scale cell culture) such as those used to produce therapeutic proteins, or antibodies. Thus, a selected shear-protectant additive (e.g., one that is effective for protecting greater than 80% of viable cells) may be used to in a large-scale manufacturing processes to produce, for example and without limitation, Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anrukinzumab, Apolizumab, Arcitumomab, Aselizumab, Atinumab, Atlizumab, Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilimumab, Tildrakizumab, Tigatuzumab, TNX-, Tocilizumab, Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and/or Zolimomab aritox.

Various other aspects and embodiments of the present disclosure, provided herein are small-scale methods for evaluating sample variations (e.g., batch-to-batch variations) of a shear-protectant additive. Methods may comprise the steps of (a) culturing cells in cell culture media in a shake flask having a volume of less than 10 L, wherein (i) the cell culture media is supplemented with a shear-protectant additive at a concentration of 0.01 g/L to 10 g/L of the cell culture media, and (ii) the cells are shaken for a period of time to produce bubbles in the media in an amount sufficient to cause a greater than 5% drop in cell viability compared to the initial cell viability; (b) measuring one or more cell performance parameters of the cultured cells and/or spent media to obtain one or more cell performance values; and (c) selecting the shear-protectant additive if the one or more cell performance values is comparable to one or more reference values. The reference values may be based on cell performance parameters of cells cultured under similar conditions in the presence of a shear-protectant additive known to be effective for protecting cells from shear damage. Alternatively, the reference values may be based on a positive control or a negative control used in the assay.

In some embodiments, methods comprise the steps of (a) culturing cells in cell culture media in a shake flask having a volume of less than 10 L, wherein (i) the cell culture media is supplemented with a shear-protectant additive at a concentration of 0.01 g/L to 10 g/L of the cell culture media, and (ii) the cells are shaken for a period of time to produce bubbles in the media in an amount sufficient to cause a greater than 5% drop in cell viability compared to the initial cell viability; (b) measuring the viability of the cultured cells; and (c) selecting the shear-protectant additive if the viability of the cultured cells drops by less than 10% as compared to the initial cell viability.

In some embodiments, methods comprise the steps of (a) culturing cells in cell culture media in a shake flask having a volume of less than 10 L, wherein (i) the cell culture media is supplemented with a shear-protectant additive at a concentration of 0.01 g/L to 10 g/L of the cell culture media, and (ii) the cells are shaken for a period of time to produce bubbles in the media in an amount sufficient to cause a greater than 5% drop in cell viability compared to the initial cell viability; (b) measuring the viability of the cultured cells; and (c) selecting the shear-protectant additive if the viability of the cultured cells is greater than 80%.

In some embodiments, methods comprise the steps of, for each of a plurality of shear-protectant additives, (a) culturing cells in cell culture media in a first shake flask having a volume of less than 10 L, wherein the cell culture media is supplemented with a first shear-protectant additive at a concentration of 0.01 g/L to 10 g/L of the cell culture media, (b) culturing cells in cell culture media in a second shake flask having a volume of less than 10 L, wherein the cell culture media is supplemented with a second shear-protectant additive at a concentration of 0.01 g/L to 10 g/L of the cell culture media, (c) shaking the cells in the first and second shake flask for a period of time to produce bubbles in the media in an amount sufficient to cause a greater than 5% drop in cell viability compared to the initial cell viability; (d) measuring one or more cell performance parameters of the cultured cells in the first and second shake flask; and (e) selecting the shear-protectant additive that is most effective for protecting cells against shear damage.

In some embodiments, the cells are mammalian cells. In some embodiments, the cells are non-mammalian cells. The cells may also be bacterial cells, insect cells, microalgae cells, yeast cells, plant cells or other cell type. In some embodiments, the cells are human cells such as, for example, human stem cells. In some embodiments, the cells are recombinant cells engineered to produce a therapeutic protein.

In some embodiments, the shake flask may be a baffled shake flask, which may be used to improve mixing and aeration as well as to generate bubbles when shaking.

In some embodiments, the volume of the shake flask may be 125 ml to 3 L. In some embodiments, the volume of the shake flask is 1 L.

In some embodiments, the shear-protectant additive is a surfactant. The surfactant may be selected from a poloxamer, a polyvinyl alcohol and a polyethylene glycol. In some embodiments, the shear-protectant additive is a poloxamer (e.g., PLURONIC® F-68, KOLLIPHOR® P-188, LUTROL® F-68), which is a nonionic triblock copolymer composed of a central hydrophobic chain of poly(propylene oxide) flanked by two hydrophilic chains of poly(ethylene oxide).

In some embodiments, the concentration of the shear-protectant additive may be 0.5 g/L to 2 g/L cell culture media.

In some embodiments, the cells may be cultured for 1 hour to 1 week. For example, the cells may be cultured for 1 day to 3 days. However, in some embodiments the cells are not cultured in the solution prior to performing the assay.

In some embodiments, the working volume of the cell culture media in the shake flask may be 10% to 30% of the volume of the shake flask.

In some embodiments, the cells may be shaken on an orbital shaker. The orbital shaker may have an orbital diameter of 19 mm to 50 mm, or 25 mm to 50 mm.

In some embodiments, the cells may be shaken at a speed of 50 rpm to 500 rpm.

In some embodiments, the cells may be cultured at a temperature of 30° C. to 40° C. In some embodiments, the cells are cultured at a CO₂ concentration of 3% to 10%. However, in some embodiments the cells are not cultured in the solution prior to performing the assay.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced herein.

### EXAMPLES

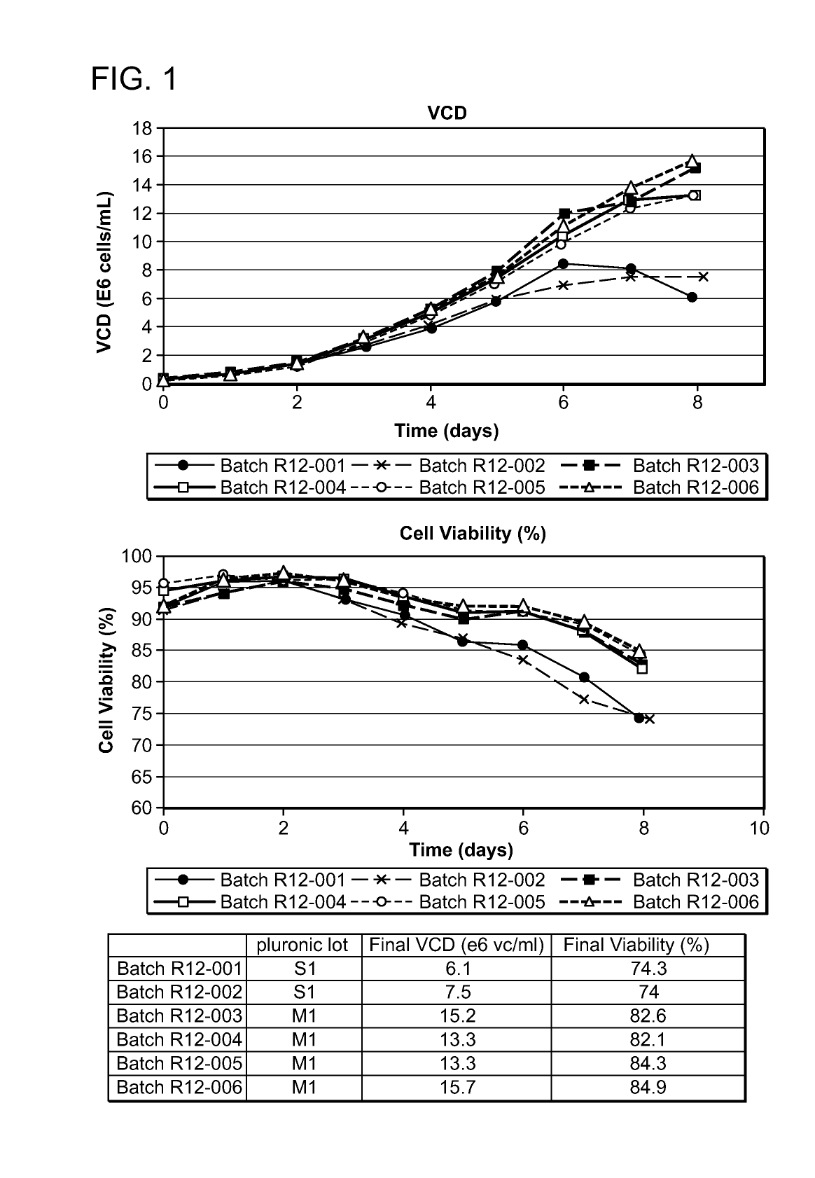

PLURONIC′ F-68 is considered a key component in cell culture media. Without it, cells cannot survive in a sparged bioreactor. Nonetheless, PLURONIC® has lot-to-lot variations, which can significantly affect cell culture performance. Mammalian cells cultured in a chemically defined media supplemented with PLURONIC® F-68 (lot S1) using a large-scale (e.g., 2000 L) bioreactor resulted in a decrease of peak viability cell density (VCD) from 15e6 vc/mL to 8e6 vc/mL, viability from 85% to 75%, and titer from 40 mg/L to 25-26 mg/L (FIG. 1). In an initial attempt to identify the cause of this decreased performance, mammalian cells were cultured in a chemically defined media supplemented with PLURONIC® F-68 from lot S1 using a 3 L sparged bioreactor. Surprisingly, this bioreactor experiment was not capable of detecting the decreased cell performance resulting from use of Pluronic F-68 from lot S1.

To provide a process for detecting variations (e.g., lot-to-lot variations) among shear-protectant additives such as PLURONIC® F-68, a cell culture system with baffled shake flasks containing air bubbles was developed, without the use of sparging or forced aeration. The following Examples are directed to the detection of batch-to-batch, or lot-to-lot, variations of PLURONIC® F-68, but methods provided herein in the various aspects and embodiments of the present disclosure are not limited to PLURONIC® F-68 and can be used to assess other shear-protectant additives (e.g., nonionic surfactants).

For the following Examples, the cell culture system was placed into an incubator at 35° C. and 5% CO₂. A vial of mammalian cells was thawed into a chemically defined media and passaged several times. The cells were then passaged in the same media supplemented with the indicated concentration of PLURONIC® F-68. The baffled shake flask size, working volume, PLURONIC® concentration, shaker orbital size, culture duration and shaking speed were adjusted to obtain desired difference among various PLURONIC® lots. All the baffled shake flasks were placed into an incubator at 35° C. and 5% CO₂.

### Example 1

Conditions—1 L baffled shake flask, 200 mL working volume, 1.5 g/L PLURONIC® F-68, 50 mm orbit shaker, 125 rpm, 3-day culture.

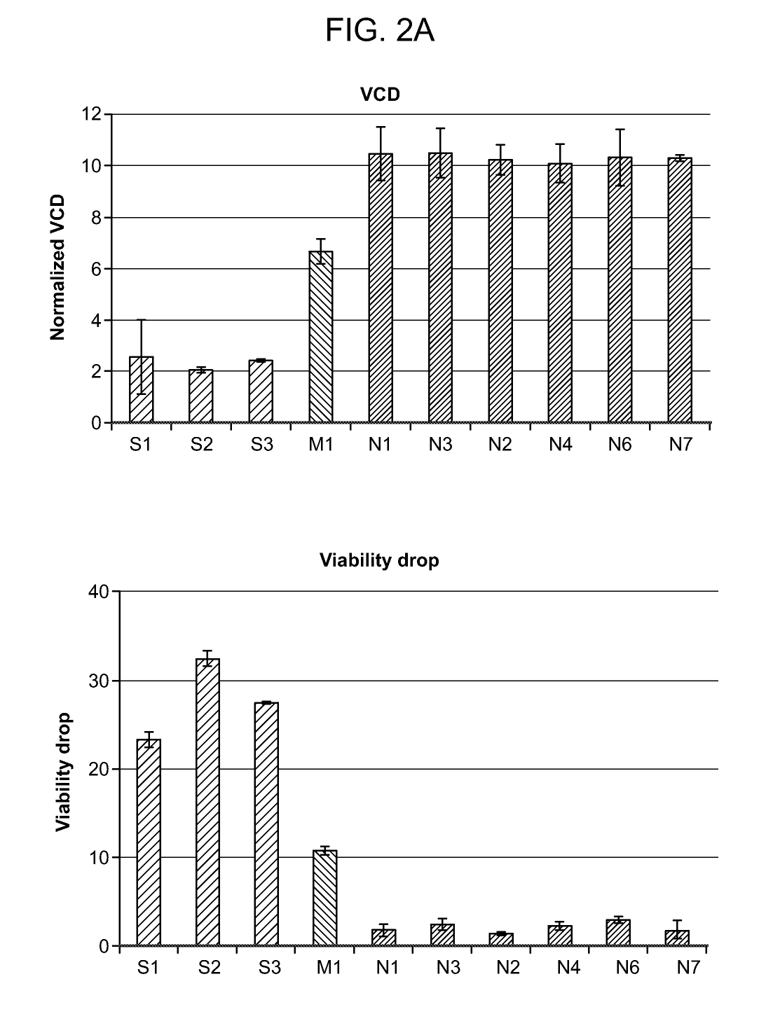

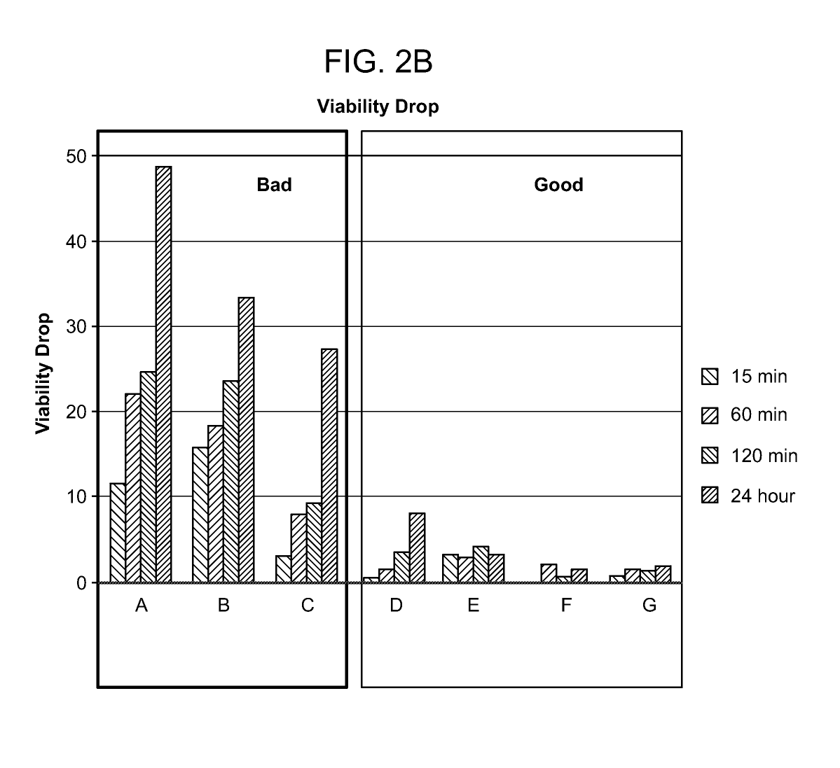

Results—Three lots (lots S1-S3), resulted in low cell growth with a large drop in viability; six lots (lots N1-N4, N6-N7) resulted in normal cell growth with a minimal drop in viability; and one lot (M1) resulted in performance between the latter two (FIG. 2A). This experiment demonstrates that the small-scale baffled shake flask cell culture system can be used to screen for lot-to-lot variations of cell culture additives such as PLURONIC®. FIG. 2B shows that the difference in viability drop between suitable and unsuitable PLURONIC® F-68 lots can be observed as quickly as 15 minutes.

### Example 2

Conditions—1 L baffled shake flask, 150 mL working volume, 1.0 g/L PLURONIC® F-68, 25 mm orbit shaker, 200 rpm, 1-day culture.

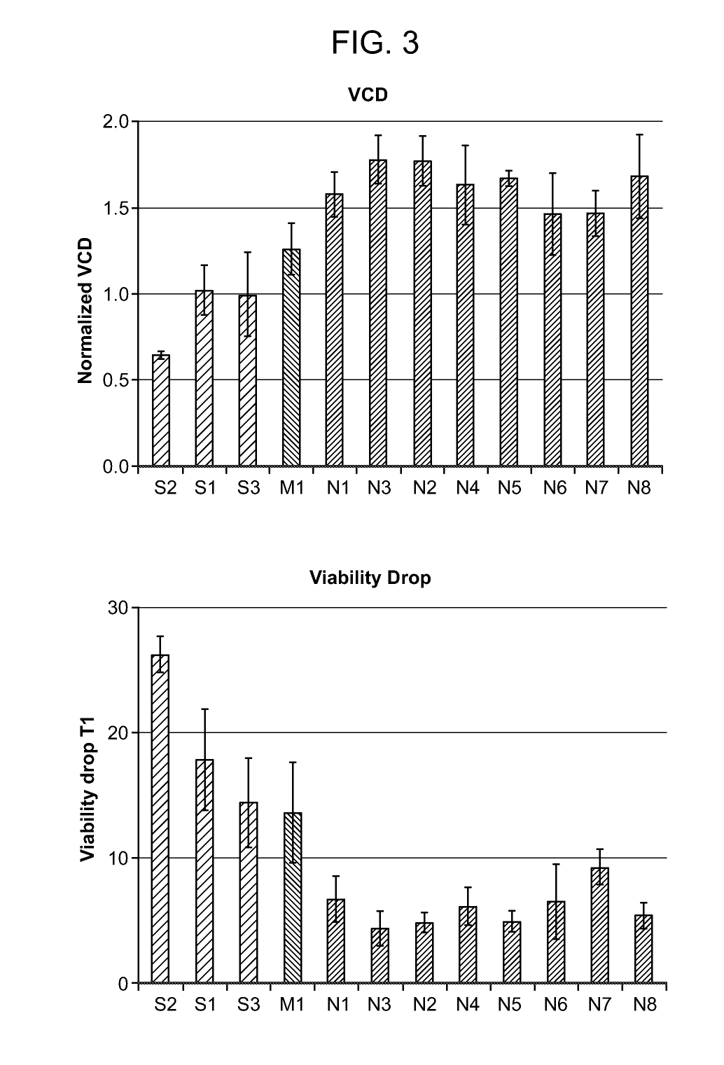

Results—Three lots (S1-S3), resulted in low cell growth with a large drop in viability; eight lots (N1-N8) resulted in normal cell growth with a minimal drop in viability; one lot (M1) resulted in performance between the latter two (FIG. 3). The results of this experiment are consistent with those of Example 1, with the added advantage of being able to detect minor differences within the N1-N8 lots and within the S1-S3 lots.

### Example 3

Conditions—Similar to those in Example 2, but with two other cell lines. The cell growth with lot N4 was used as a control (100%) to eliminate the cell line difference.

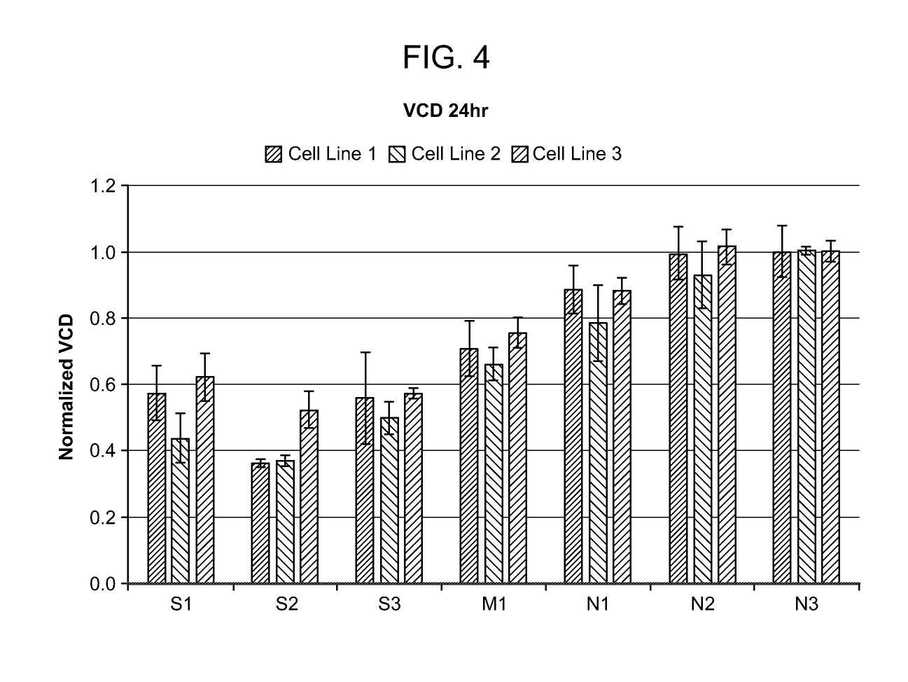

Results—The small-scale baffled shake flask cell culture system can be used to detect PLURONIC® variation using difference cell lines, and all three cell lines have similar sensitivity to PLURONIC® variations (FIG. 4).

### Example 4

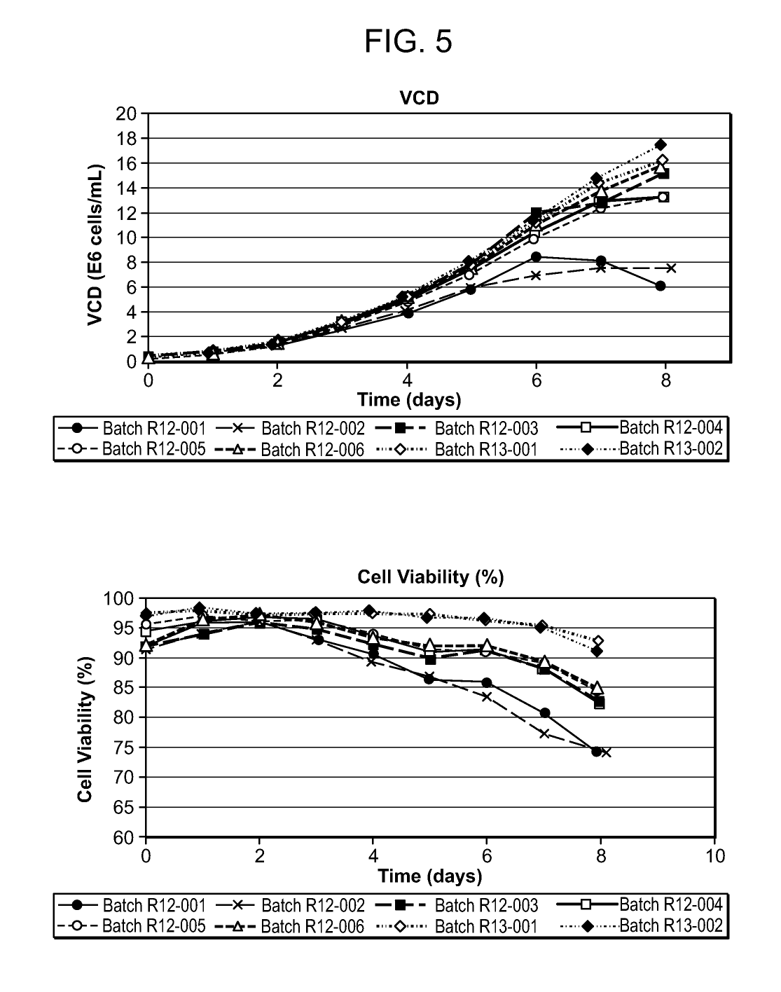

The N6 lot, which showed suitable performance in the baffled shake flask cell culture system, was used in a large-scale (e.g., 2000 L) bioreactor system. The cell performance results from two batches are shown in FIG. 5 as Batch R13-001 and Batch R13-003. Results showed that using this lot of PLURONIC® resulted in high cell growth, high viability (>90%), and high titer (53 mg/L vs. 40 mg/L).

### Example 5

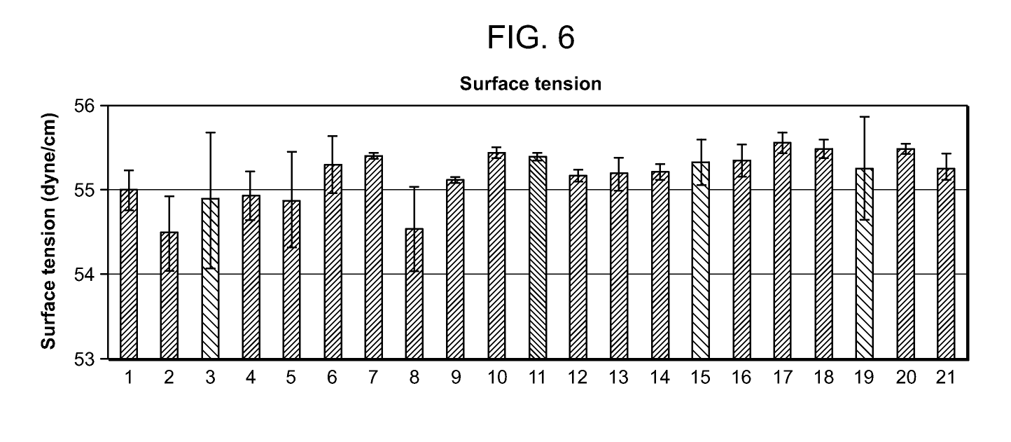

Surface tension is an easy and common way to evaluate properties of surfactants. However, as shown in FIG. 6, surface tension does not correlate with cell culture performance. The difference among various samples is not significant. Shear-protectant additives (e.g., surfactants), especially common ones used in cell culture process, can facilitate foam formation under sparging or shaking conditions (FIG. 7, left). Foam stability is closely related to the properties of surfactants. FIGS. 8-10 show, as discussed in greater detail below, that the foam layer dissipation time for “suspicious” lots of PLURONIC® F-68 (e.g., those suspected of being less protective of shear damage), some of which are unsuitable (or “bad”) lots and some of which are suitable (or “good”) lots. The foam layer dissipation time for unsuitable lots is longer in comparison to suitable lots (e.g., those effective at protective cells against shear damage).

A 200 mL solution of WPU (water for pharmaceutical use) and 1.5 g/L of one of several lots of PLURONIC® F-68 and 200 ppm antifoaming agent (e.g., DOW CORNING® antifoam Q7-2587 30% Simethicone Emulsion USP) was shaken overnight at 125 rpm in a 1 L baffled shake flask (50 mm orbit shake base, 35° C., 5% CO₂, and 70% humidity). The shaking was then stopped, and the duration of time between the stop and foam dissipating was measured and compared. Three suspicious lots (1-3) and one intermediate lot (4) had significantly longer dissipation times than three suitable lots (5-7), which correlated with viability drop profiles (FIG. 8).

### Example 6

A 200 mL solution of WPU, 1.5 g/L of one of several lots of PLURONIC® F-68, and 200 ppm anti-foam Q7-2587 was shaken overnight at 125 rpm in a 1 L baffled shake flask (50 mm orbit shake base, room temperature, no control on CO₂ and humidity). The shaking was then stopped, and the duration of time between the stop and foam dissipating was measured and compared. One suspicious lot (1) had a significantly longer dissipation time than five suitable lots (8, 9, 10, 11 and 6), which correlated with viability drop profiles (FIG. 9).

### Example 7

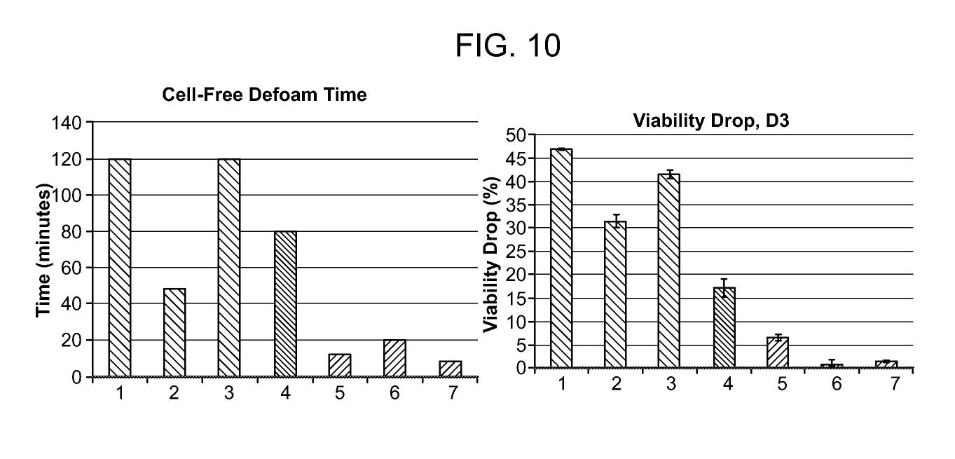

A 150 mL solution of WPU, 1.0 g/L of one of several lots of PLURONIC® F-68, and 200 ppm anti-foam Q7-2587 was shaken overnight at 200 rpm in a 1 L baffled shake flask (25 mm orbit shake base, room temperature, no control on CO₂ and humidity). The shaking was then stopped, and the duration of time between the stop and foam dissipating was measured and compared. Three suspicious lots (1-3) and one intermediate lot (4) had significantly longer dissipation times than three suitable lots (5-7), which correlated with viability drop profiles (FIG. 10).

### Example 8

Based on the foam stability data, it was clear that there are differences among various PLURONIC® F-68 lots in terms of surfactant composition and property at foam layer. The difference might be small and hard to detect under normal conditions. The process of foam generation can enrich or fractionate surfactants on bubble surface and foam layer, which enlarge the differences in surfactant raw material to a level that can be detected by analytical methods such as size-exclusion chromatography (SEC) with refractive index detection.

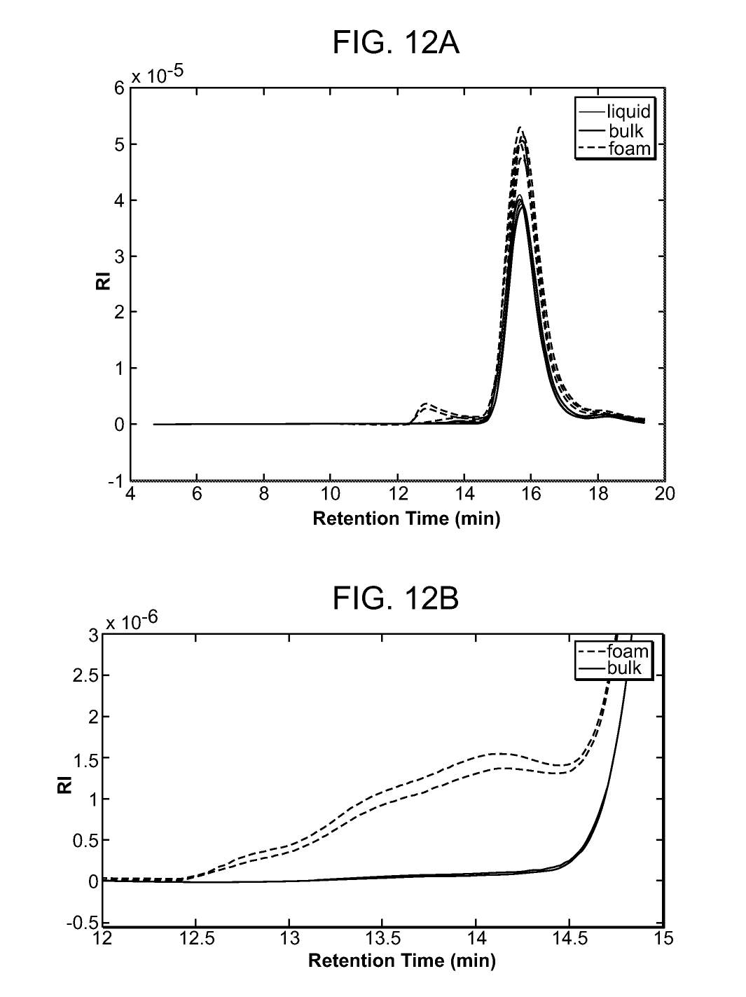

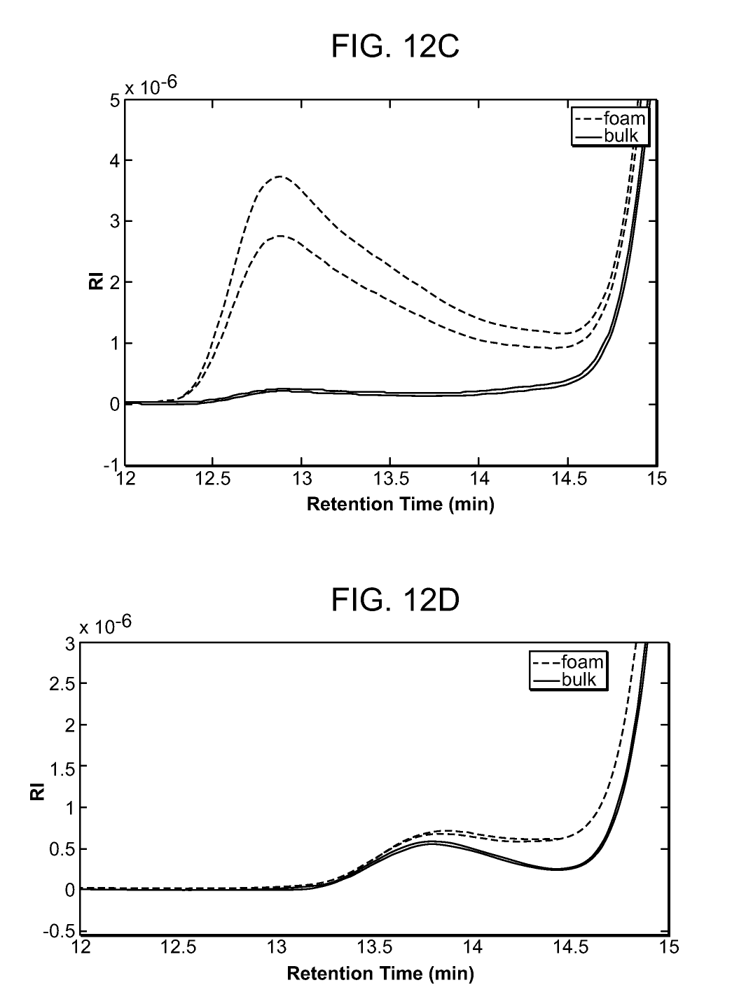

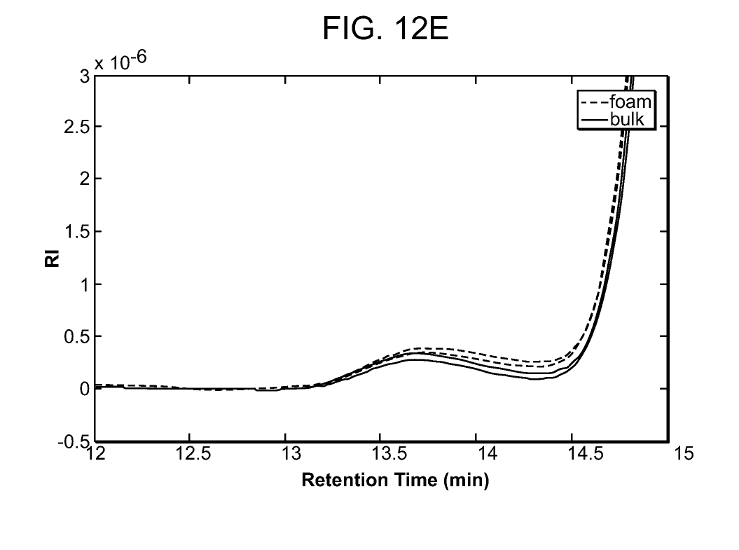

A 200 mL solution of WPU, 1.5 g/L of one of several lots of PLURONIC F-68 was shaken overnight at 125 rpm in a 1 L baffled shake flask (50 mm orbit shake base, room temperature, no control on CO₂ and humidity). The shaking was then stopped. Bulk liquid in the shake flask (e.g., liquid without foam) was removed carefully with a pipette to let foam layer dissipate (e.g., liquefy). Samples from bulk liquid, liquefied foam, and solution control (before the shaking) were collected and measured by size exclusion chromatography. Suspicious/unsuitable lots of PLURONIC® F-68 showed significantly more peak area in high molecular weight regions (<14.7 min), particularly in foam samples (FIG. 11A). The difference was at high molecular weight (MW) region (<14.7 mins). Suspicious/unsuitable PLURONIC® F-68 lots (FIGS. 11B, 11C, 12B and 12C) and intermediate lots (FIG. 11D) showed large separation between foam and bulk samples and larger peak areas of high MW species (also referred to as components) in foam samples. Suitable PLURONIC® F-68 lots (FIGS. 12D, 12E, 11F) had smaller separation between foam and bulk samples. Both had small peak area of high MW species. One of the PLURONIC® F-68 lots (FIG. 12D) had a slightly larger peak area at high MW region relative to other two suitable lots (FIGS. 11E and 11F), which corresponded to the slightly higher viability drop shown in FIG. 10 (lot 5).

The detailed conditions of SEC test are listed below.
Column: TSKgel G2000 SWXL (8 mm ID×40 cm, 5 μm).
Guard: TSKgel Guard SuperSW (4.6 mm ID×4.5 cm, 4 μm).
Mobile Phase: 10 mM Sodium Chloride in 10% Methanol.
Flow rate: 0.5 mL/min.
Load: 400 μg.
Triplicate injection per sample.

### Example 9

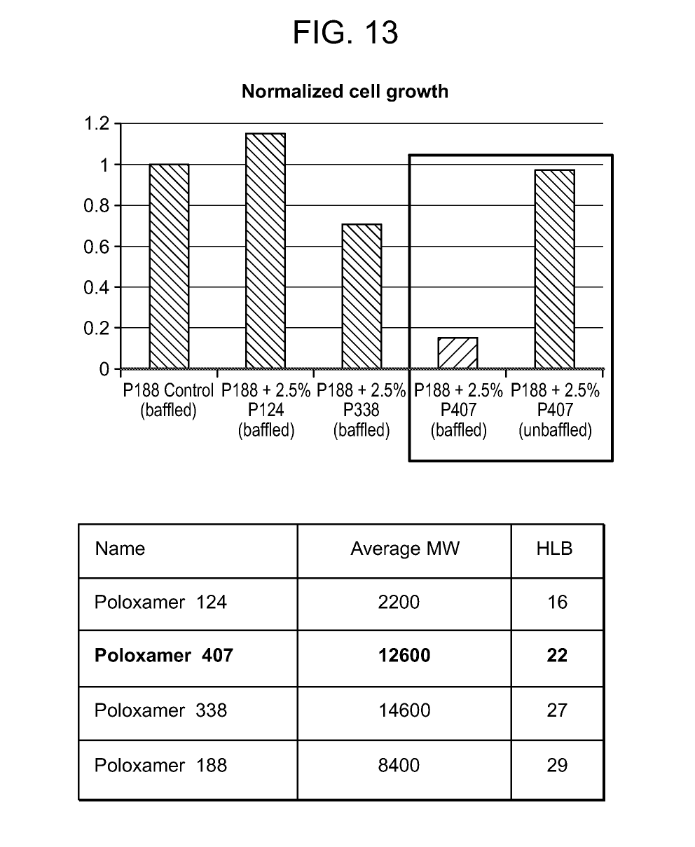

To investigate whether poor performance of unsuitable lots of shear-protectant additive (e.g., lots suspected of having an adverse effect on cell performance) was due to the existence of hydrophobic components in the additive, a small percentage (˜2.5%) of poloxamer 124, poloxamer 407 or poloxamer 338, each having a different molecular weight and hydrophobicity, was added to a suitable lot of poloxamer 188 (e.g., a lot known not to have an adverse effect on cell performance). FIG. 13 shows that, using a baffled shake flask system of the present disclosure, cell growth dropped significantly when cells were grown in the presence of both poloxamer 188 and poloxamer 407 relative to cell growth in the presence of poloxamer 188 only. Adverse effects were not observed when an unbaffled shake flask system was used. Results showed that even a small proportion of other hydrophobic molecules can adversely affect the efficacy of poloxamer 188 for protecting cells from bubble/shear damage (FIG. 13). Poloxamer 407 has a higher molecular weight and a higher hydrophobicity (or a low hydrophilic-lipophilic balance (HLB) value) relative to poloxamer 188. Similarly, poloxamer 338, which also has a higher molecular weight and a higher hydrophobicity (low HLB) relative to poloxamer 188, lowers the performance of poloxamer 188 by ˜30%, (FIG. 13). Poloxamer 124, however, which has a higher relative hydrophobicity (low HLB), but a lower relative molecular weight, did not lower the performance of poloxamer 188 (FIG. 13). Thus, in some instances, both molecular weight and hydrophobicity may be used as parameters for assessing the efficacy of shear-protectant additives.

The data shown in FIG. 13 is consistent with foam/SEC results, which showed that unsuitable lots contain high molecular weight components enriched in the foam layer. Even though hydrophobicity was not measured directly, enrichment of the high molecule weight components in the foam layer suggests that those high molecular weight components are highly hydrophobic.

### Example 10

In order to further investigate whether the poor performance of unsuitable shear-protectant additive lot can be attributed to the presence of highly hydrophobic components as suggested in foam enrichment experiment (Example 8) and in the demonstration study (Example 9), a large preparative size exclusion chromatography (SEC) column (e.g., 320 ml volume) was used to separate the HMW fraction from the remaining fractions of a sample. Column information is shown in Table 1 below.

TABLE 1

| Material Name | Supplier | Part Number |
|---|---|---|
| HPLC Vials | Waters | Total recovery vials |
| SEC Column | TOSOH | Catalog No: 08540, TSKgel G2000 |
| (Analytical) | Biosci- | SWXL(7.8 mm ID × 30 cm, 5 μm) |
| | ence | |
| Guard Column | TOSOH | Catalog No: 18762, TSKgel Guard |
| (Analytical) | Biosci- | SuperSW (4.6 mm ID × 3.5 cm, 4 um) |
| | ence | |
| SEC Mobile | N/A | 10 mM Sodium Chloride + 10% Methanol |
| Phase A | | |
| C3 RP Column | Agilent | Poroshell 300SB-C3 2.1 × 75 mm, 5 μm |
| RP Mobile | N/A | H2O + 0.1% TFA |
| Phase A | | |
| RP Mobile | N/A | 90% Acentonitrile (ACN) + 0.1% TFA |
| Phase B | | |
| Preparative SEC | | HiPrep 26/60 Sephacryl S-100 HR |
| Column | | (26 mm ID × 60 cm, 25 μm-75 μm) |

100 mg/ml samples of an unsuitable poloxamer 188 lot (Lot #1) were prepared for fractionation. The load volume was set to 10.0 ml, while the flow rate was set to 1.0 ml/min. 10 ml fractions were collected using a fraction collector until the end of 1 column volume (CV). This process was repeated several times to provide enough material for other testing and characterization.

After the determination of the appropriate fractions using high performance liquid chromatography (HPLC)-SEC with an analytical column, the different fractions were pooled together. The samples were then frozen using liquid nitrogen and then placed in the lyophilizer for 4 days until no more solvent was present in the beakers. After the lyophilization step was complete, the samples were dissolved in water to the desired concentration.

The hydrophobicity of prepared samples was tested using reverse phase (RP)-HPLC with a C3 column. The poloxamer molecule does not have an absorbance in the UV-Vis region and does not fluoresce; therefore, a charged aerosol detector (CAD) had to be used to detect the poloxamer components eluting from the column. The column temperature was set to 40° C. with a flow rate of 0.5 ml/min. Run time set to 35 minutes. 40 μl of sample were injected each run.

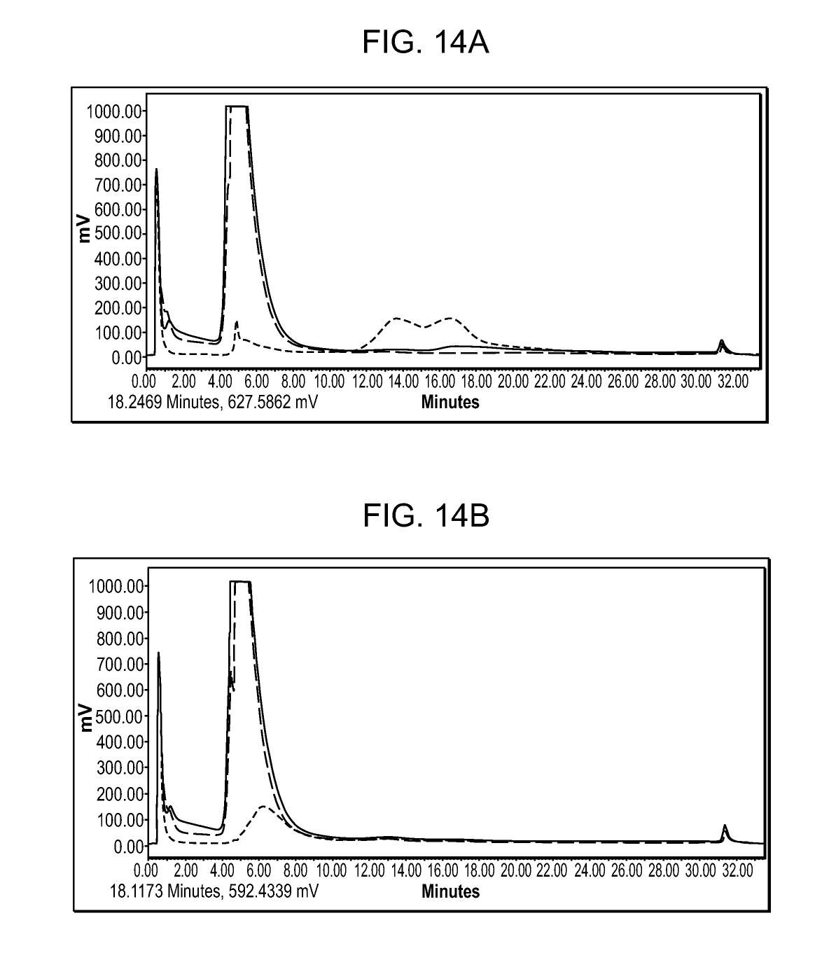

FIG. 14A shows fraction 11 (HMW, shown in light gray), 17 (Main peak, shown in black), and 22 (Main peak, shown in dark gray). Fraction 11, containing HMW components, shows highly hydrophobic peaks that elute between 12-28 minutes while fractions 17 and 22 contain only the main peak which elutes early in the chromatogram at 5 minutes into the run. This indicates that more hydrophobic components did exist in unsuitable lot, in this case, in HMW faction. By comparison, FIG. 14B shows that a suitable performance lot does not have any high hydrophobic components eluted in 12-18 minutes region.

Nuclear Magnetic Resonance (NMR) spectroscopy was used to assess any structural differences in each of the fractions. NMR spectra were acquired on fractionated poloxamer samples before the lyophilization step. Five fractions were selected to be tested and the percent of oxyethylene content was calculated using the USP pharmacopeia protocol for poloxamer weight percent oxyethylene. Normally, poloxamer 188 contains 81.8% oxyethylene+/−1.9%. The fractionated samples were first dried using a speed vacuum technique and reconstituted 1:1 in deuterated chloroform. The final sample concentration remained the same because 1 ml of fractured sample was dried and dissolved in 1 ml of deuterated chloroform. NMR spectra were acquired by averaging 1024 scans and a D1 relaxation of 9 seconds. 32 dummy scans were acquired first to make sure the protons are in steady state. Poloxamer regularly has a methyl peak at 1.14 ppm and several backbone peaks at 3.2 ppm-4.0 ppm. The poloxamer peaks were integrated following the USP pharmacopeia protocol. It was found that the earlier fractions containing HMW from the SEC analysis have a lower percentage of oxyethylene (70.8% vs. normal at 81.8%+/−1.9%). Oxyethylene is known to be the hydrophilic part of the poloxamer molecule and, therefore, a decrease in that percentage would make the molecule more hydrophobic. Thus, in some instances, presence of a low percentage of oxyethylene (e.g., less than 75%) may be indicative of a shear-protectant additive having poor cell performance.

The hydrophobic component (in this case in HMW region) from the unsuitable lot (Lot #1) was then added to a suitable lot (Lot #2) at a ratio of 0.9%. A 3-day baffled shake flask system was used to test the impact on cell culture performance of the suitable lot of shear-protectant additive. The addition of hydrophobic components (in this case in HMW region) to the suitable lot resulted in a cell viability drop of 21%, which is significantly higher than the control (2% cell viability drop), which shows that hydrophobic components (in this case in HMW region) from the suspicious lot has a negative impact on cell performance, even at a very low concentration.

In sum, it was demonstrated that unsuitable lots contain high hydrophobic components and/or high molecular weight. Both RP-HPLC and NMR data support the conclusion that unsuitable lots are more hydrophobic than expected in normal poloxamer 188 samples, in some instances, the level of high hydrophobic components is very low and hard to detect. The negative impact of highly hydrophobic components from an unsuitable lot was shown using a cell culture test with SEC fractionation.

### Example 11

### Size Exclusion Chromatography (SEC)—Different Lots of Poloxamer 188

Various lots of poloxamer 188 were tested using SEC and compared against their performance. FIG. 15 shows three chromatograms highlighting the different peaks. The HMW peak eluting in the region from 12-14.5 minutes is split into two peaks labeled Peak 1 and Peak 2. The main peak elutes at 15 minutes while the low molecular weight (LMW) peak elutes at 18 minutes. The top chromatogram shows a high performance poloxamer lot, the middle chromatogram shown a poloxamer lot with medium performance while the last chromatogram on the bottom shown a low performance lot.

FIG. 16 indicates that the low performance poloxamer lot contains specie of HMW (labeled Peak 1) that is not present in the high performance lot and is present in a small amount in the medium performance lot. From this figure, one can observe a dose response correlating the HMW with low performance.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for evaluating sample variations of a shear-protectant additive, the method comprising the steps of:
(a) producing, in a solution that comprises viable cells and a shear-protectant additive at a concentration of about 0.01 g/L to about 10 g/L solution, bubbles in an amount sufficient to cause a greater than about 5% drop in cell viability relative to initial cell viability;
(b) measuring one or more cell performance parameters of the cells to obtain one or more cell performance values; and
(c) selecting the shear-protectant additive if the one or more cell performance values is comparable to one or more reference values.

2. The method of claim 1, further comprising shaking the solution in a shake flask.

3. The method of claim 2, wherein the shake flask is a baffled shake flask.

4. The method of claim 2 or 3, wherein the volume of the shake flask is less than 10 L.

5. The method of claim 4, wherein the volume of the shake flask is about 125 ml to about 3 L.

6. The method of claim 5, wherein the volume of the shake flask is about 1 L.

7. The method of any one of claims 2-6, wherein the working volume of the solution in the shake flask is about 10% to about 30% of the volume of the shake flask.

8. The method of any one of claims 1-7, wherein the solution comprises buffer.

9. The method of any one of claims 1-8, wherein the solution comprises cell culture media.

10. The method of any one of claims 1-9, wherein the shear-protectant additive is a surfactant.

11. The method of claim 10, wherein the surfactant is selected from a poloxamer, a polyvinyl alcohol and a polyethylene glycol.

12. The method of claim 11, wherein the surfactant is a poloxamer.

13. The method of any one of claims 1-12, wherein the concentration of the shear-protectant additive is about 0.5 g/L to about 2 g/L solution.

14. The method of any one of claims 1-13, wherein the cells are mammalian cells.

15. The method of any one of claims 1-14, further comprising culturing the viable cells in the solution.

16. The method of claim 15, wherein the cells are cultured for about 15 minutes to about 1 week.

17. The method of claim 15 or 16, wherein the cells are cultured at a temperature of about 30° C. to about 40° C.

18. The method of any one of claims 15-17, wherein the cells are cultured at a CO₂ concentration of about 3% to about 10%.

19. A method for evaluating sample variations of a shear-protectant additive, the method comprising the steps of:
(a) producing, in a solution that comprises viable cells and a shear-protectant additive at a concentration of about 0.01 g/L to about 10 g/L solution, bubbles in an amount sufficient to cause a greater than about 5% drop in cell viability relative to initial cell viability;
(b) measuring the viability of the cells; and
(c) selecting the shear-protectant additive if the viability of the cells drops by less than 10% as compared to the initial cell viability.

20. A method for evaluating sample variations of a shear-protectant additive, the method comprising the steps of:
(a) producing, in a solution that comprises viable cells and a shear-protectant additive at a concentration of about 0.01 g/L to about 10 g/L solution, bubbles in an amount sufficient to cause a greater than about 5% drop in cell viability relative to initial cell viability;
(b) measuring the viability of the cells; and
(c) selecting the shear-protectant additive if the viability of the cells is greater than 80%.

21. A method for evaluating sample variations of a shear-protectant additive, the method comprising the steps of:
(a) producing, in a first solution that comprises viable cells and a shear-protectant additive at a concentration of about 0.01 g/L to about 10 g/L solution, bubbles in an amount sufficient to cause a greater than about 5% drop in cell viability relative to initial cell viability;
(b) producing, in a second first solution that comprises viable cells and a shear-protectant additive at a concentration of about 0.01 g/L to about 10 g/L solution, bubbles in an amount sufficient to cause a greater than about 5% drop in cell viability relative to initial cell viability;
(c) measuring one or more cell performance parameters of the cells in the first and second solution; and
(d) selecting the shear-protectant additive that is most effective for protecting cells against shear damage.

22. A method for evaluating sample variations of a shear-protectant additive, the method comprising the steps of:
(a) producing a foam layer in a solution that comprises a shear-protectant additive at a concentration of about 0.01 g/L to about 10 g/L solution;
(b) measuring a duration of time during which the foam layer dissipates to obtain a dissipation time; and
(c) selecting the shear-protectant additive if the dissipation time is comparable to a reference value.

23. The method of claim 22, wherein the volume of the foam layer is about 20% to about 200% of the total volume of the solution.

24. The method of claim 23, wherein the volume of the foam layer is about 100% of the total volume of the solution.

25. The method of any one of claims 22-24, wherein the solution further comprises an antifoaming agent.

26. The method of any one of claims 22-25, further comprising shaking the solution in a shake flask.

27. The method of claim 26, wherein the shake flask is a baffled shake flask.

28. The method of claim 26 or 27, wherein the volume of the shake flask is less than 10 L.

29. The method of claim 28, wherein the volume of the shake flask is about 125 ml to about 3 L.

30. The method of claim 29, wherein the volume of the shake flask is about 1 L.

31. The method of any one of claims 26-30, wherein the working volume of the solution in the shake flask is about 10% to about 30% of the volume of the shake flask.

32. The method of any one of claims 22-31, wherein the solution comprises water.

33. The method of any one of claims 22-32, wherein the solution comprises buffer.

34. The method of any one of claims 22-33, wherein the shear-protectant additive is a surfactant.

35. The method of claim 34, wherein the surfactant is selected from a poloxamer, a polyvinyl alcohol and a polyethylene glycol.

36. The method of claim 35, wherein the surfactant is a poloxamer.

37. The method of any one of claims 22-36, wherein the concentration of the shear-protectant additive is about 0.5 g/L to about 2 g/L solution.

38. The method of any one of claims 22-37, wherein the reference value is a dissipation time obtained from a control solution containing a shear-protectant additive effective for protecting cells against shear damage.

39. The method of any one of claims 22-37, wherein the reference value is 40 minutes, and the shear-protectant additive is selected if the dissipation time is less than 40 minutes.

40. The method of any one of claims 22-37, wherein the reference value is 30 minutes, and the shear-protectant additive is selected if the dissipation time is less than 30 minutes.

41. The method of any one of claims 22-37, wherein the reference value is 20 minutes, and the shear-protectant additive is selected if the dissipation time is less than 20 minutes.

42. A method for evaluating sample variations of a shear-protectant additive, the method comprising the steps of:
(a) producing a foam layer in a test solution that comprises a sample of shear-protectant additive at a concentration of about 0.01 g/L to about 10 g/L test solution;
(b) collecting a liquefied foam layer sample from the test solution;
(c) producing a size exclusion chromatography (SEC) chromatogram of the liquefied foam layer sample;
(d) comparing the high molecular weight peak of the SEC chromatogram to a reference value; and
(e) selecting the shear-protectant additive if the high molecular weight peak of the SEC chromatogram is comparable to the reference value.

43. The method of claim 42, wherein the reference value is a pre-determined value.

44. The method of claim 42 or 43, wherein the reference value is based on a high molecular weight peak of a SEC chromatogram from a control sample of a solution containing a sample of a shear-protectant additive known to be effective for protecting cells against shear damage.

45. The method of any one of claims 42-44, wherein the control sample is from the bulk layer of the test solution.

46. The method of any one of claims 42-45, wherein the test solution is a cell-free solution.

47. A method for evaluating sample variations of a shear-protectant additive, the method comprising the steps of:
(a) producing a foam layer in a first test solution that comprises a first sample of shear-protectant additive at a concentration of about 0.01 g/L to about 10 g/L test solution;
(b) producing a foam layer in a second test solution that comprises a second sample of shear-protectant additive at a concentration of about 0.01 g/L to about 10 g/L test solution;
(c) collecting first and second liquefied foam layer samples from the first and second test solutions, respectively,
(d) producing a first and second size exclusion chromatography (SEC) chromatogram of the first and second liquefied foam layer samples, respectively;
(e) comparing the high molecular weight peak of the first and second SEC chromatograms to each other; and
(f) selecting the shear-protectant additive with the smallest high molecular weight peak.

48. The method of claim 47, wherein the second test solution comprises a control solution containing a sample of a shear-protectant additive known to be effective for protecting cells against shear damage.

49. The method of claim 47 or 48, wherein the test solution is a cell-free solution.

50. A method for evaluating sample variations of a shear-protectant additive, the method comprising the steps of:
(a) producing a foam layer in a plurality of test solutions that each comprise a sample of respective shear-protectant additives at a concentration of about 0.01 g/L to about 10 g/L test solution;
(b) collecting a liquefied foam layer sample from respective test solutions;
(c) producing a size exclusion chromatography (SEC) chromatogram of respective liquefied foam layer samples;
(d) comparing the high molecular weight peaks of respective SEC chromatograms; and
(e) selecting the shear-protectant additive with the smallest high molecular weight peak.

51. The method of claim 50, wherein the test solution is a cell-free solution.

52. A method for evaluating the suitability of a shear-protectant additive for use in large-scale cell culture, the method comprising:
assaying a sample of a poloxamer for the presence of a marker of unsuitability, and identifying the preparation as suitable for use in large-scale cell culture if the marker of unsuitability is not present.

53. A method for evaluating the suitability of a shear-protectant additive for use in large-scale cell culture, the method comprising:
assaying a sample of a poloxamer for the presence of a marker of unsuitability, and identifying the preparation as unsuitable for use in large-scale cell culture if the marker of unsuitability is present.

54. The method of claim 52 or 53, wherein the poloxamer is a poloxamer 188.

55. The method of claim 54, wherein the marker of suitability is a component having a molecular weight of greater than 12 kDa.

56. The method of claim 54 or 55, wherein the marker of suitability is a hydophilic-lipophilic balance value of less than 29.

57. A method for evaluating efficacy of a shear-protectant additive for preventing shear damage to cells, the method comprising detecting in a sample of a shear-protectant additive a high molecular weight components and/or a highly hydrophobic components, and identifying the sample as an unsuitable sample.

58. The method of claim 57, wherein the shear-protectant additive is poloxamer 188 and the high molecular weight components has a molecular weight of greater than 12 kDa.

59. The method of claim 57 or 58, wherein the shear-protectant additive is poloxamer 188 that has a hydrophilic-lipophilic balance (HLB) value of less than 29.

60. A method for evaluating efficacy of a shear-protectant additive for preventing shear damage to cells, the method comprising assaying a sample of a shear-protectant additive for a high molecular weight components and/or a highly hydrophobic components, and identifying the sample as a suitable sample if a high molecular weight components and/or a highly hydrophobic components is not detected.

61. A method for evaluating efficacy of poloxamer 188 for preventing shear damage to cells, the method comprising determining the proportion of hydrophilic chains and hydrophobic chains in poloxamer copolymers obtained from a sample of poloxamer 188, and then identifying the sample as unsuitable if the hydrophilic chains constitutes less than 80% of the copolymers.

62. The method of claim 61, wherein the sample is identified as unsuitable if the hydrophilic chains constitutes less than 78% of the copolymers.

63. The method of claim 62, wherein the sample is identified as unsuitable if the hydrophilic chains constitutes less than 75% of the copolymers.
